(19) United States
(12) Patent Application Publication
(10) Pub. No.: US 2023/0295457 A1
(43) Pub. Date: Sep. 21, 2023

(54) SURFACE TREATMENT LIQUID AND SURFACE TREATMENT METHOD

(71) Applicant: TOKYO OHKA KOGYO CO., LTD., Kanagawa (JP)

(72) Inventors: Takahiro SENZAKI, Kanagawa (JP); Hisato SHIMIZU, Kanagawa (JP)

(21) Appl. No.: 18/042,828

(22) PCT Filed: Jul. 28, 2021
(86) PCT No.: PCT/JP2021/027979

§ 371 Date: Feb. 24, 2023

(30) Foreign Application Priority Data
Aug. 27, 2020 (JP) 2020-143884

(51) Int. Cl. C09D 139/04; C09D 147/00

(52) U.S. Cl. CPC C09D 139/04; C09D 147/00

(57) ABSTRACT

A surface treatment liquid which adhesiveness of a component that achieves a surface treatment effect is good, and a desired surface treatment effect is easily obtained, and a surface treatment method using the surface treatment liquid are provided.

A surface treatment liquid including a resin (A) and a solvent (S), in which the resin (A) includes a constituent unit (a1) derived form a compound having a hydrophilic group and an ethylenic double bond and a constituent unit (a2) derived from a compound a nitrogen-containing heterocyclic group, an ethylenic double bond, and a specific type of polar group is used.

### TECHNICAL FIELD

The present invention relates to a surface treatment liquid and a surface treatment method in which the surface treatment liquid is used.

### BACKGROUND ART

In order to modify the properties of surfaces of a variety of articles, surface treatments have been thus far carried out using a variety of surface treatment liquids. As surface modification, particularly, adjustment of hydrophilicity of the surfaces of articles has been significantly demanded, and a number of chemicals and surface treatment liquids for hydrophilization or hydrophobization have been proposed. A surface treatment of an object using a chemical or surface treatment liquid for hydrophilization of hydrophobization forms a coating on the surface of the object and hydrophilizes or hydrophobizes the surface of the object.

As such chemicals and surface treatment liquids, for example, a surface treatment agent including a copolymer of monomer including at least acrylamide monomer and mono(meth)acrylate monomer as a component for hydrophilic property (refer to Patent Document 1) and a surface treatment agent containing a block copolymer including a polyvinyl alcohol resin block having mercapto group and a polyanionic resin block, and polyacrylic acid (refer to Patent Document 2) have been proposed. The polyanionic resin block disclosed in Patent Document 2 is a block in which a polymerizable monomer having at least one carboxylic group and/or sulfonic acid group in one molecule is polymerized.

Patent Document 1: Japanese Patent No. 5437523

Patent Document 2: Japanese Unexamined Patent Application, Publication No. 2009-126948

### DISCLOSURE OF THE INVENTION

### Problems to be Solved by the Invention

However, the resins included in the surface treatment agents disclosed in Patent Document 1,Patent Document 2, and the like does not necessarily have sufficient adhesiveness to the surface of object to be treated. As a result, conventional surface treatment agents disclosed in Patent Literature 1, Patent Literature 2, and the like, have problems in that it is sometimes difficult to obtain the desired surface treatment effect, or the surface treatment effect easily decreases due to peeling of the resin from the surface of the object to be treated.

The present invention has been made in consideration of the above-described problem, and an objective of the present invention is to provide a surface treatment liquid which adhesiveness of a component that achieves a surface treatment effect is good, and a desired surface treatment effect is easily obtained, and a surface treatment method using the surface treatment liquid. Means for Solving the Problems

The present inventors found that it was possible to solve the above-mentioned problem by a surface treatment liquid including a resin (A) and a solvent (S), in which the resin (A) includes a constituent unit (a1) derived form a compound having a hydrophilic group and an ethylenic double bond and a constituent unit (a2) derived from a compound a nitrogen-containing heterocyclic group, an ethylenic double bond, and a specific type of polar group, and completed the present invention. In more detail, the present invention provides the followings.

A first aspect of the present invention is a surface treatment liquid including a resin (A), and a solvent (S), in which the resin (A) includes a constituent unit (a1) derived from a compound having a hydrophilic group and an ethylenic double bond and a constituent unit (a2) derived from a compound represented by formula (a-2).

(R^{A3}—R^{A2})ₙ—X—R^{A1} (a-2)

In the formula (a-2), R^{A2} is an organic group having one or more ethylenic double bond. R^{A2} is a single bond, or an alkylene group having 1 or more and 10 or less carbon atoms. R^{A3} is a hydrogen atom or a polar group selected from amino group, carboxylic group, mercapto group, hydroxy group, and cyano group. n is 1 or 2. X is a nitrogen-containing heterocyclic group having valency of n+1. When n is 1, R^{A3} is the polar group. When n is 2, at least one of R^{A3}s are the polar group.

A second aspect of the present invention is a surface treatment method to a surface of the object to be treated, including forming a coating film on the surface of the object to be treated by applying the surface treatment liquid according to the first aspect.

### Effects of the Invention

According to the present invention, it is possible to provide a surface treatment liquid which adhesiveness of a component that achieves a surface treatment effect is good, and a desired surface treatment effect is easily obtained, and a surface treatment method using the surface treatment liquid.

### PREFERRED MODE FOR CARRYING OUT THE INVENTION

### «Surface Treatment Liquid»

A surface treatment liquid includes a resin (A) and a solvent (S). This treatment liquid can adjust hydrophilicity of a surface of the object to be surface treated. Hereinafter, with respect to the surface treatment liquid, an essential component and an optional component will be described.

### <Resin (A)>

A resin (A) includes a constituent unit (a1) derived from a compound a hydrophilic group and an ethylenic double bond for a purpose of adjusting hydrophilicity of the surface of the object to be treated by surface treatment. It should be noted that the resin (A) absolutely exhibits a hydrophilic property. However, depending on a relative relationship between the hydrophilicity of the resin (A) and the hydrophilicity of the surface of the object to be treated, there is a case that the hydrophilicity of the surface of the object to be treated after surface treatment is lower than that of the surface of the object to be treated before surface treatment. The resin (A) includes a constituent unit (a2) derived from a compound represented by the following formula (a-2) for a purpose of good adhesion of the resin (A) to the surface of the object to be treated. The resin (A) may include other constituent unit (a3) other than the constituent unit (a1) and the constituent unit (a2) as long as the objective of the present invention is not impaired.

(R^{A3}—R^{A2})ₙ—X—R^{A1} (a-2)

In the formula (a-2), R^{A1} is an organic group having one or more ethylenic double bond. R^{A2} is a single bond, or an alkylene group having 1 or more and 10 or less carbon atoms. R^{A3} is a hydrogen atom or a polar group selected from amino group, carboxylic group, mercapto group, hydroxy group, and cyano group. n is 1 or 2. X is a nitrogen-containing heterocyclic group having valency of n+1. When n is 1, R^{A3} is the polar group. When n is 2, at least one of R^{A3}s are the polar group.

It should be noted that amino group, carboxylic group, and hydroxy group are correspond to the hydrophilic group. Therefore, when the constituent unit (a2) is derived from the compound represented by the formula (a-2) which is a compound having the 1 or more ethylenic unsaturated double bond as R^{A1}, and having amino group, carboxylic group, mercapto group, or hydroxy group as R^{A3}, the constituent unit (a2) is also the constituent unit (a1). In this case, a homopolymer of 1 monomer selected from, or copolymer of the 2 or more selected from the compounds represented by the formula (a-2) which are compounds having the 1 or more ethylenic unsaturated double bond as R^{A1}, and having amino group, carboxylic group, mercapto group, or hydroxy group as R^{A3} is defined as the resin (A) including the constituent unite (a1) and the constituent unit (a2). In view of easily obtaining the desired effect by using the above surface treatment liquid, the resin (A) preferably includes the constituent unit (a1) and the constituent unit (a2) as constituent units which differ from each other. More specifically, the resin (A) preferably includes the constituent unit (a1) not corresponding to the constituent unit (a2), and the constituent unit (a2).

[Constituent Unit (a1)]

The constituent unit (a) is a constituent unit having a hydrophilic group and an ethylenic double bond.

The hydrophilic group is not particularly limited as long as the hydrophilic group is a functional group that has thus far been recognized as a hydrophilic group by a person skilled in the art and can be appropriately selected from such functional groups. Specific examples of the hydrophilic group include a polyoxyalkylene group such as polyoxyethylene group, a polyoxypropylene group, and a polyoxyalkylene group consisting of a block or random combination of oxyethylene group(s) and oxypropylene group(s), a carboxy group, a primary amino group, a secondary amino group, a hydroxy group, a phosphonic acid group, a phosphinic acid group, a sulfonic acid group, and the like. An organic group including these groups is preferable as the hydrophilic group.

As the hydrophilic group, a group represented by the following formula (ai) is preferable in view of superior hydrophilic effect.

—NH—R¹ (ai)

In the formula (ai), R¹ is an alkyl group having 1 or more and 4 or less carbon atoms substituted with one or more groups selected from the group consisting of amino group, sulfonic acid group, phosphonic acid group, and hydroxy group, or hydrogen atom. With respect to R¹, amino group corresponds to a cationic group, and sulfonic acid group and phosphonic acid group corresponds to an anionic group.

Specific examples of the hydrophilic group represented by the formula (ai) include the amino group and the groups represented by the following formulas.

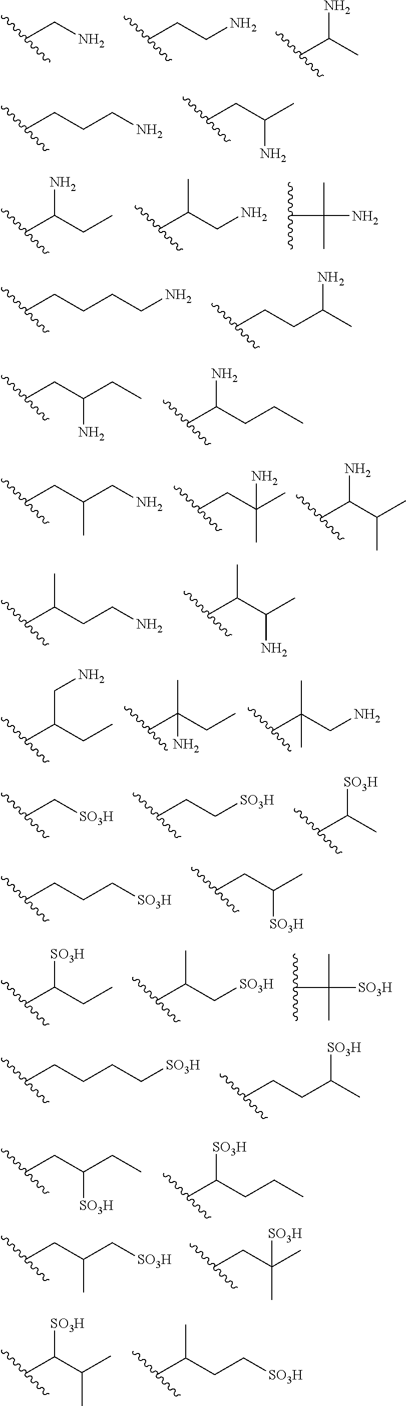

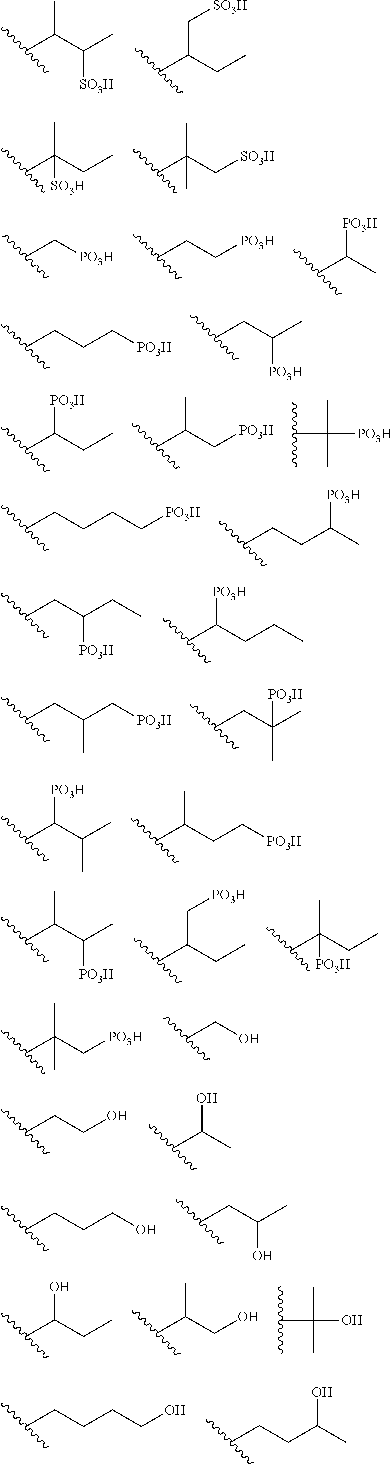

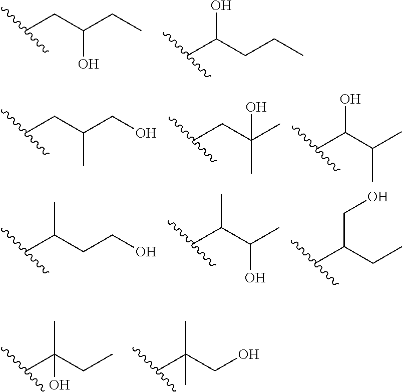

Among the above-described specific examples of the hydrophilic group represented by the formula (ai), the following groups are exemplified as the more preferable group.

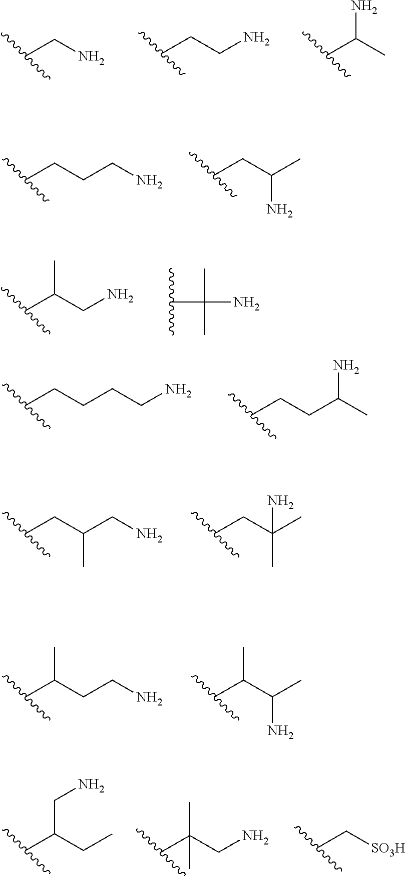

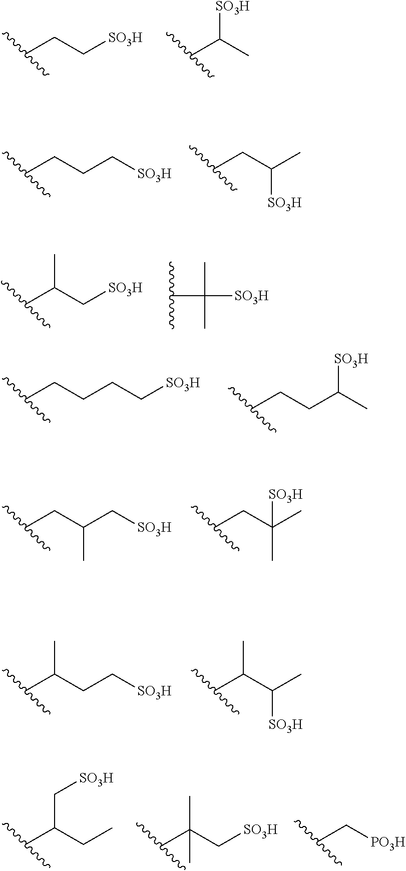

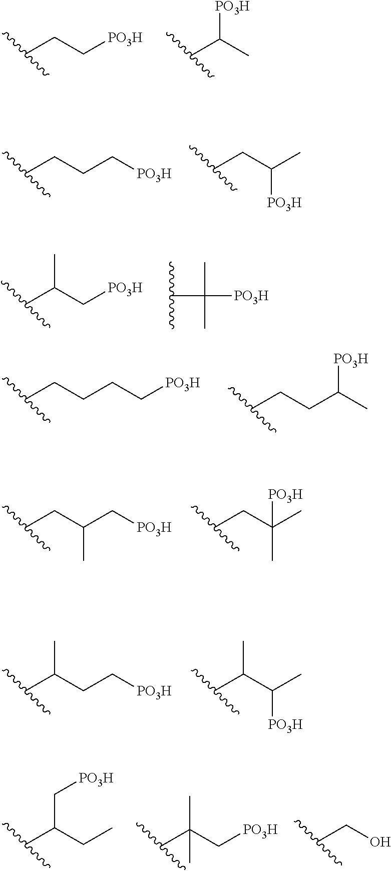

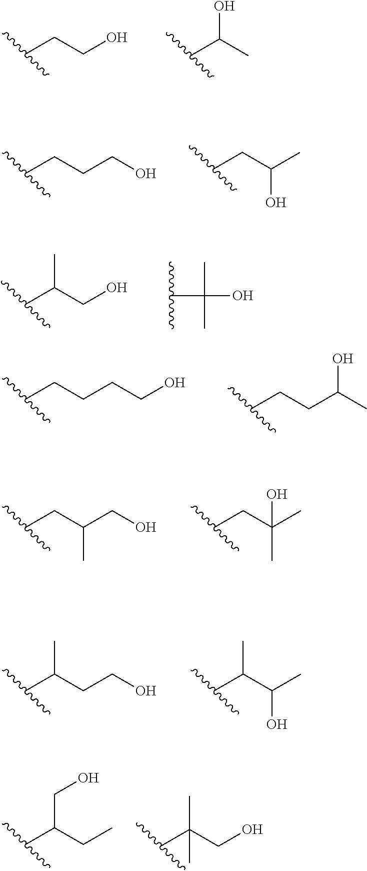

Among the above-described specific examples of the hydrophilic group represented by the formula (ai), the following groups are exemplified as the particularly preferable group.

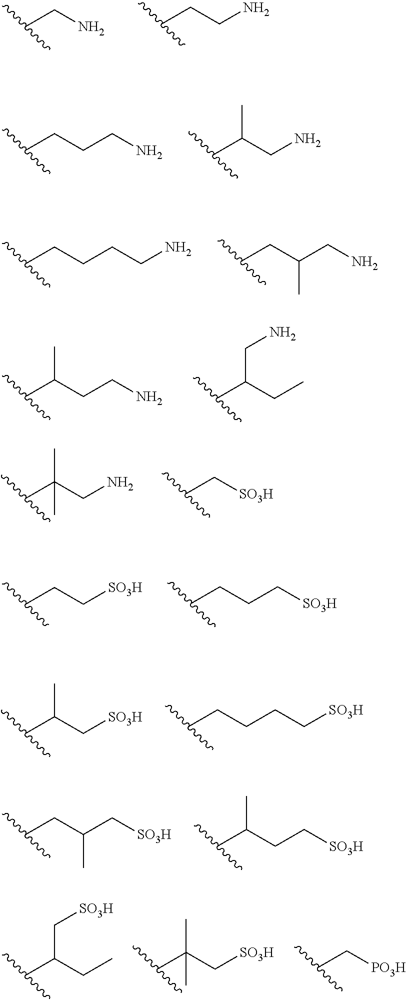

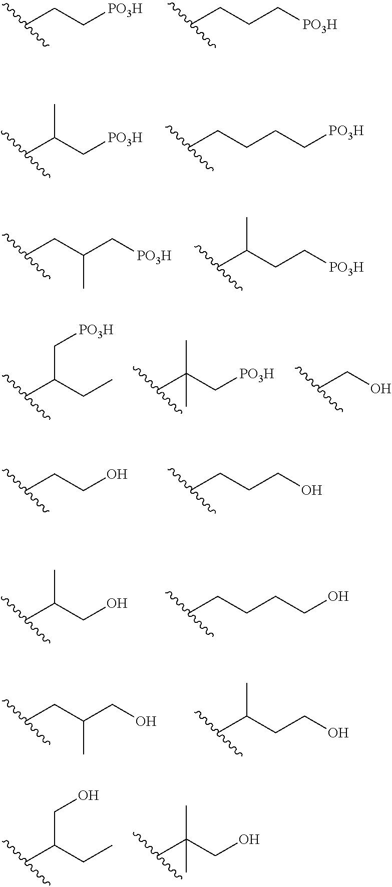

As described above, the compound giving the constituent unit (a1) has the hydrophilic group and the ethylenic double bond. Therefore, the compound giving the constituent unit (a1) has a group having the ethylenic double bond. Examples of the group having the ethylenic double bond include alkenyl groups such as vinyl group, 1-propenyl group, 2-n-propenyl group (allyl group), 1-n-butenyl group, 2-n-butenyl group, and 3-n-butenyl group, acryloyloxy group, methacryloyloxy group, acryloylamino group, and methacryloylamino group.

In view of polymerizability and ease of synthesis and availability, the compound giving the constituent unit (a1) is preferably a (meth)acrylic amide compound represented by the following formula (a1-a).

CH₂═CR²—CO—NH—R¹ (a1-a)

In the formula (a1-a), R¹ is an alkyl group having 1 or more and 4 or less carbon atoms substituted with one or more groups selected from the group consisting of amino group, sulfonic acid group, phosphonic acid group, and hydroxy group, or hydrogen atom. R² is hydrogen atom or methyl group.

In the formula (a1-a), R¹ is the same as described above.

Suitable specific examples of the constituent unit (a1) derived from the compound represented by the formula (a1-a) include the following units a1-a-1 to a1-a-5. Among the following units, units a1-a-1 to a1-a-4 are more preferred.

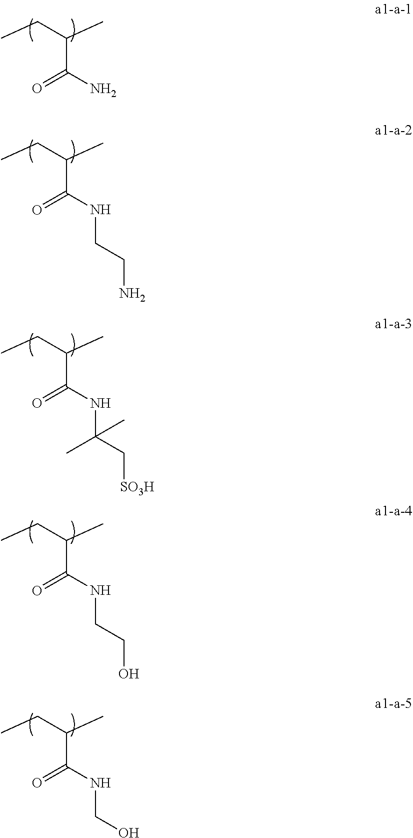

In addition, the resin (A) preferably includes a constituent unit derived from a betaine monomer having a cationic group, an anionic group, and an ethylenic double bond as the constituent unit (a1). Both of the cationic group and the anionic group act as the hydrophilic group. There is a case that the surface-treated surface of the object to be treated comes into contact with a cleaning liquid including a large amount of anions with hydrophobic groups or cations with hydrophobic groups. In a case where a resin in the surface treatment liquid has only an anionic group such as a carboxy group, a carboxylate group, a sulfonic acid group or a sulfonate group as the hydrophilic group, this hydrophilic group may stop acting as a hydrophilic group due to an interaction with a cation having a hydrophobic group. In addition, in a case where the resin (A) in the surface treatment liquid has only a cationic group such as a quaternary ammonium group as the hydrophilic group, the cationic group may stop acting as a hydrophilic group due to an interaction with an anion having a hydrophobic group. However, in case that the resin (A) has both of the cationic group and the anionic group as the hydrophilic group, an action as the hydrophilic group of any one of the cationic group and the anionic group can be maintained, and hydrophobicity of the surface of the object to be treated is hardly decrease, when the surface-treated surface of the object to be treated comes into contact with a cleaning agent including a large amount of cations having hydrophobic groups or cleaning agent including a large amount of anions having the hydrophobic group.

A number of the cationic group and a number of the anionic group in the betaine monomer giving the constituent unit (a1) are not particularly limited. The number of the cationic group and the number of the anionic group in the betaine monomer giving the constituent unit (a1) are preferably the same. The number of the cationic groups and the number of the anionic groups in the polymerizable betaine compound are each preferably one since the synthesis or procurement of the betaine monomer giving the constituent unit (a1) is easy.

In the betaine monomer giving the constituent unit (a1), for example, a group having the ethylenic double bond, the cationic group, and the cationic group are preferably bonded, if necessary, via a bridging group, in this order.

The cationic group is preferably a cationic group that is a quaternary nitrogen cation. The anionic group is preferably sulfonic acid anion group, phosphonic acid anion group, or carboxylic acid anion group.

As the group having the ethylenic double bond in the betaine monomer giving the constituent unit (a1), alkenyl groups such as vinyl group, 1-propenyl group, 2-n-propenyl group (allyl group), 1-n-butenyl group, 2-n-butenyl group, and 3-n-butenyl group are exemplified. Among these groups, vinyl group and 2-n-propenyl group (ally group) are preferred. A number of the ethylenic double bond in the betaine monomer giving the constituent unit (a1) is not particularly limited, and preferably 1 or 2.

As the betaine monomer giving the constituent unit (a1), compounds represented by the following formula (a1-i) or formula (a1-ii) are preferred. The betaine monomers represented by the following formula (a1-i) or formula (a1-ii) include the cationic group including N⁺ and the anionic group as R. Both of the cationic group and the anionic group act as the hydrophilic group.

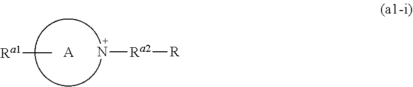

In the formula (a1-i), R^{al} is a hydrocarbon group including the ethylenic double bond. Rae is a divalent hydrocarbon group having 1 or more and 10 or less carbon atoms. R is the anionic group. Ring A is a heterocycle.

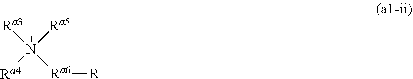

In the formula (a1-ii), R^{a3}, R^{a4}, and R^{a5} are each independently a hydrocarbon group having the ethylenic double bond or a hydrocarbon group having 1 or more and 10 or less. At least one of R^{a3}, R^{a4}, and R^{a5} are the hydrocarbon group having an ethylenic unsaturated double bond. R^{a6} is a divalent hydrocarbon group having 1 or more and 10 or less carbon atoms. R is an anionic group.

In the formula (a1-i), as the hydrocarbon group including the ethylenic double bond as R^{a1}, alkenyl groups such as vinyl group, 1-propenyl group, 2-n-propenyl group (allyl group), 1-n-butenyl group, 2-n-butenyl group, and 3-n-butenyl group are exemplified.

In the formula (a1-i), as the divalent hydrocarbon group as Rae, an alkylene group, an arylene group, and a group which is a combination of the alkylene group and the arylene group are preferred. Suitable specific examples of the alkylene group as Rae include methylene group, ethane-1,2-diyl group, propane-1,3-diyl group, propane-1,2-diyl group, butane-1,4-diyl group, butane-1,4-diyl group, pentane-1,5-diyl group, hexane-1,6-diyl group, heptane-1,7-diyl group, octane-1,8-diyl group, nonane-1,9-diyl group, and decane-1,10-diyl group.

In the formula (a1-i), the heterocycle as ring A may be an aromatic heterocycle or an aliphatic heterocycle. As the aromatic heterocycle, a ring in which any one of the nitrogen atom(s) in a nitrogen-containing aromatic heterocycle such as imidazole ring, pyrazole ring, 1,2,3-triazole ring, 1,2,4-triazole ring, pyridine ring, pyrimidine ring, pyridazine ring, and pyrazine is quaternized is exemplified. As the aliphatic heterocycle, a ring in which any one of the nitrogen atom(s) in a nitrogen-containing heterocycle such as pyrrolidine ring, piperidine ring, and piperazine ring is quaternized is exemplified.

In the formula (a1-ii), as the hydrocarbon group including the ethylenic unsaturated double bond, alkenyl groups such as vinyl group, 1-propenyl group, 2-n-propenyl group (allyl group), 1-n-butenyl group, 2-n-butenyl group, and 3-n-butenyl group are exemplified.

In the formula (a1-ii), as the hydrocarbon group as R^{a3} to R^{a5}, an alkyl group, an aryl group, and aralkyl group are exemplified, and alkyl group is preferred. The hydrocarbon group as R^{a3} to R^{a5} may have a substituent. The substituent which the hydrocarbon group as R^{a3} to R^{a5} may have is not particularly limited as long as the objective of the present invention is not impaired. Examples of the substituent include a halogen atom, a hydroxy group, an alkoxy group having 1 or more and 4 or less carbon atoms, an acyl group having 2 or more and 4 or less carbon atoms, an acyloxy group having 2 or more and 4 or less carbon atoms, an amino group, an alkylamino group substituted with one or two alkyl group having 1 or more and 4 or less carbon atoms, and the like. Suitable specific examples of the alkyl group as R^{a3} to R^{a5} include methyl group, ethyl group, n-propyl group, isopropyl group, n-butyl group, isobutyl group, sec-butyl group, tert-butyl group, n-pentyl group, n-hexyl group, n-heptyl group, n-octyl group, 2-ethylhexyl group, n-nonyl group, and n-decyl group.

In the formula (a1-ii), as the divalent hydrocarbon group as R^{a6}, an alkylene group, an arylene group, and a group which is a combination of the alkylene group and the arylene group are exemplified, and the alkylene group is preferred. Suitable specific examples of the alkylene group as R^{a6} include methylene group, ethane-1,2-diyl group, propane-1,3-diyl group, propane-1,2-diyl group, butane-1,4-diyl group, butane-1,4-diyl group, pentane-1,5-diyl group, hexane-1,6-diyl group, heptane-1,7-diyl group, octane-1,8-diyl group, nonane-1,9-diyl group, and decane-1,10-diyl group.

As the betaine monomer which has the sulfonic acid anion group as the anionic group, in view of ease of synthesis and availability, monomers represented by the following formula (a1-iii) or the formula (a1-iv) are preferred.

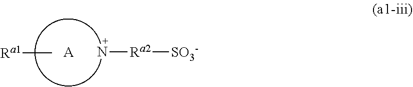

In the formula (a1-iii), R^{a1}, R^{a2}, and ring A are the same as R^{a1}, R^{a2}, and ring A in the formula (a1-i).

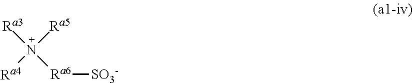

In the formula (ai-iv), R^{a3}, R^{a4}, R^{a5}, and R^{a6} are the same as R^{a3}, R^{a4}, R^{a5}, and R^{a6} in the formula (a1-ii).

As the monomers represented by the formula (ai-iii) or the formula (ai-iv), monomers represented by the following formula (a1-v), (a1-vi), or (a1-vii) are exemplified.

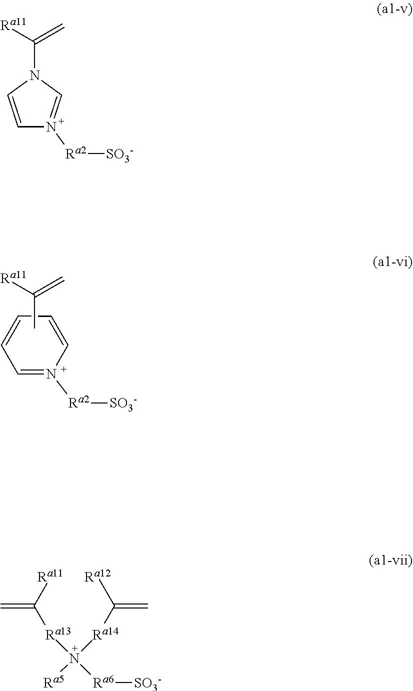

In the formula (a1-v), (a1-vi), and (a1-vii), R^{a2} is the same as R^{a2} in the formula (a1-iii). R^{a5} and R^{a6} are the same as R^{a5} and R^{a6} in the formula (a1-iv). R^{a11} and R^{a12} are each independently hydrogen atom or methyl group. R^{a13} and R^{a14} are each independently a single bond or an alkylene group having 1 or more and 4 or less carbon atoms.

In the formula (a1-v), (a1-vi), and (a1-vii), as the alkylene group having 1 or more and 4 or less carbon atoms as R^{a13} and R^{a14}, methylene group, ethane-1,2-diyl group, propane-1,3-diyl group, propane-1,2-dyl group, and butane-1,4-diyl group are exemplified.

As the betaine monomers having the phosphonic acid anion group or the carboxylic acid anion group as the anionic group, monomers represented by the formula (a1-ii) or the formula (a1-iv) described above, and monomers which the sulfonic acid anion group (—SO₃⁻) is replaced with the phosphonic acid anion group (—(PO₃)²⁻) or the carboxylic acid anion group (—COO⁻) in the monomers represented by the formula (a1-vii) are exemplified.

Specific examples of the betaine monomer represented by the formula (a1-i) or the formula (a1-ii) include monomers which the sulfonic acid anion group (—SO₃⁻) is replaced with the phosphonic acid anion group (—(PO₃)²⁻) or the carboxylic acid anion group (—COO⁻) in the following compounds.

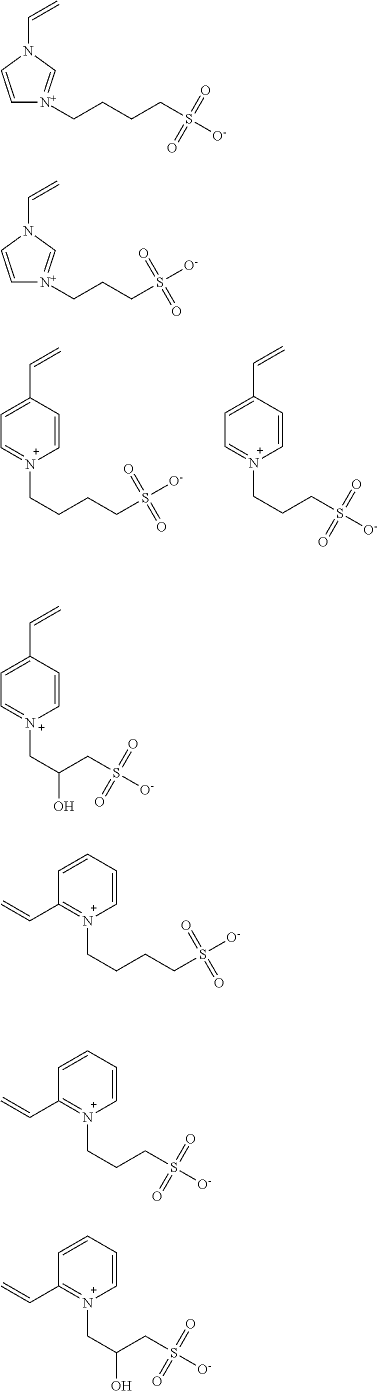

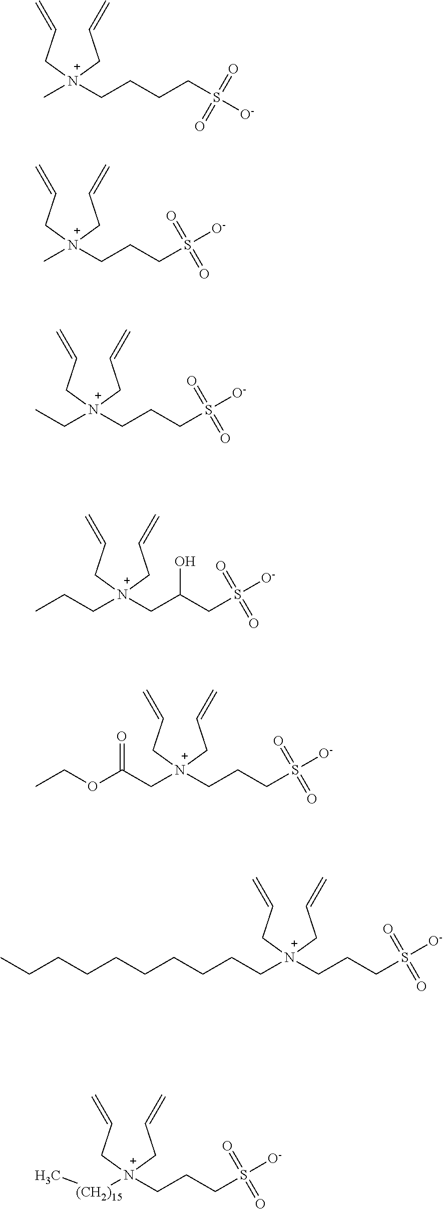

The betaine monomer represented by the formula (a1-i) or the formula (a1-ii) can be synthesized by known reactions. For example, the betaine monomer represented by the formula (a1-i) or the (a1-ii) can be obtained by reacting a compound having a group having the ethylenic unsaturated double bond and a group that can form the cationic group with a compound having the anionic group. Specifically, for example, a compound represented by the formula (a1-iii) is obtained by reacting the following compound with a sultone in a solvent. As the sultone, a sultone having 4-membered or more and 10-membered or less ring is exemplified, and 1,3-propane sultone, and 1,4-butane sultone are preferred.

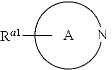

In the formula, R^{a1} is the same as R^{a1} in the above formula (a1-i), and ring A is a heterocycle.

In addition, a compound represented by the following formula (a1-viii) is also preferred as the betaine monomer giving the constituent unit (a1). The betaine monomer represented by the formula (a1-viii) includes a cationic group including N⁺ and the anionic group as R^{a20}. Both of the cationic group and the anionic group act as the hydrophilic group.

CH₂═CR^{a15}—CO—NH—R^{a16}—N⁺(R^{a17})(R^{a18})—R^{a19}—R^{a20} (a1-viii)

In the formula (a1-viii), R^{a15} is hydrogen atom or methyl group. R^{a16} and R^{a19} are each independently a divalent hydrocarbon group having 1 or more and 10 or less carbon atoms. R^{a17} and R^{a18} are each independently an optionally substituted hydrocarbon group having 1 or more and 10 or less carbon atoms. R^{a20} is sulfonic acid anion group (—SO₃⁻), phosphonic acid anion group (—(PO₃)²⁻), or carboxylic acid anion group (—COO⁻).

In the formula (a1-viii), as the divalent hydrocarbon group as R^{a16} and R^{a19}, an alkylene group, an arylene group, and a group which is a combination of the alkylene group and the arylene group are exemplified, and the alkylene group is preferred. Suitable specific examples of the alkylene group as R^{a16} and R^{a19} include methylene group, ethane-1,2-diyl group, propane-1,3-diyl group, propane-1,2-diyl group, butane-1,4-diyl group, butane-1,4-diyl group, pentane-1,5-diyl group, hexane-1,6-diyl group, heptane-1,7-diyl group, octane-1,8-diyl group, nonane-1,9-diyl group, and decane-1,10-diyl group.

In the formula (a1-viii), as the hydrocarbon group as R^{a17} and R^{a18}, an alkyl group, an aryl group, and an aralkyl group are exemplified, and the alkyl group is preferred. The hydrocarbon group as R^{a17} and R^{a18} may have a substituent. The substituent which the hydrocarbon group as R^{a17} and R^{a18} may have is not particularly limited as long as the objective of the present invention is not impaired. Examples of the substituent includes a halogen atom, a hydroxy group, an alkoxy group having 1 or more and 4 or less carbon atoms, an acyl group having 2 or more and 4 or less carbon atoms, an acyl oxy group having 2 or more and 4 or less carbon atoms, an amino group, an alkylamino group substituted with 1 or 2 alkyl groups having 1 or more and 4 or less carbon atoms, and the like. Suitable specific examples of the alkyl group as R^{a17} and R^{a18} include methyl group, ethyl group, n-propyl group, isopropyl group, n-butyl group, isobutyl group, sec-butyl group, tert-butyl group, n-pentyl group, n-hexyl group, n-heptyl group, n-octyl group, 2-ethylhexyl group, n-nonyl group, and n-decyl group.

In the formula (a1-viii), R²⁰ is sulfonic acid anion group (—SO₃⁻), phosphonic anion group (—(PO₃)²⁻) or carboxylic acid anion group (—COO⁻), and the sulfonic acid anion group (—SO₃⁻) is preferred.

Suitable examples of the N-substituted (meth)acrylic amide represented by the formula (a1-viii) include the following compounds. In the following formulas, R^{a15} is hydrogen atom or methyl group.

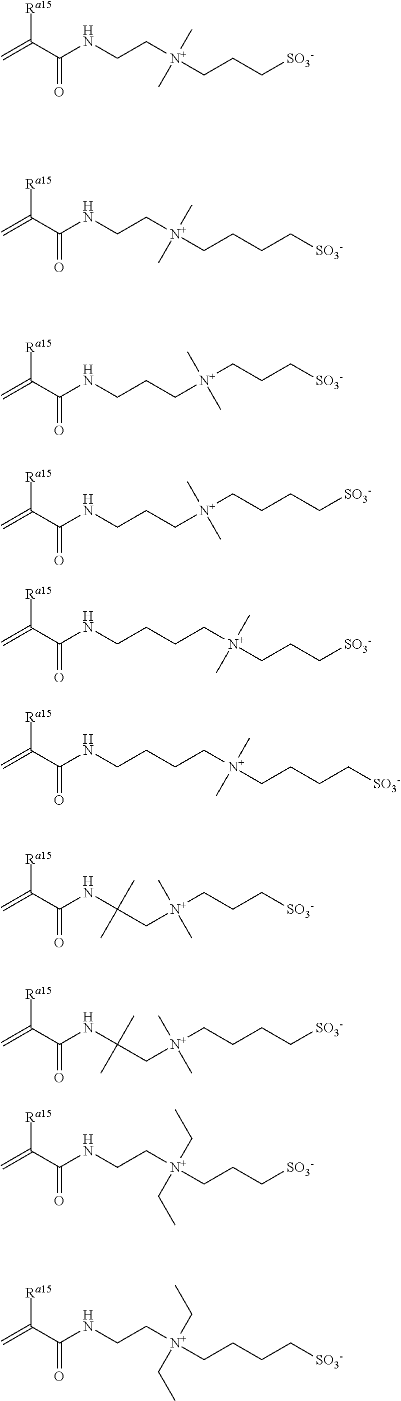

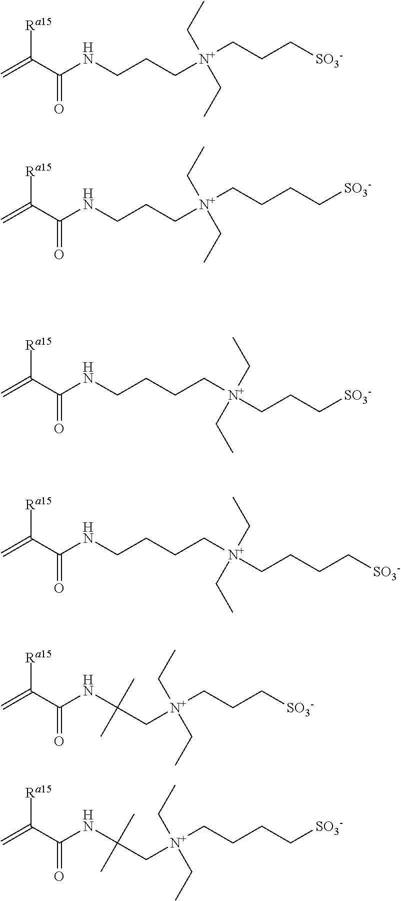

The constituent unit (a1) may be a constituent unit derived from a non-betaine monomer having an anionic group, a functional group capable of forming an anion, a cationic group, or a functional group capable of forming a cation. These functional groups in the resin (A) provide hydrophilicity to the object to be treated.

For example, cation derived from the cationic group is preferably a cation including N⁺, C⁺, B⁺, P⁺, and the like, and more preferably a cation including N. As the cationic group, from the viewpoint that the resin (A) is easily available, and good surface treatment effect is easily obtained, a cyclic or non-cyclic amino group or a quaternary ammonium salt group is preferred.

As the non-betaine monomer having the above cationic group or the functional group capable of forming cation, for example, a compound represented by the following formula (a1-ix) is preferred.

CH₂═CR^{a21}—(CO)ₚ—R^{a22} (a1-ix)

In the formula (a1-ix), R^{a21} is hydrogen atom or methyl group. R^{a22} is a group represented by —Y—R^{a23}—R^{a24} or amino group. Y is —O— or —NH—. R^{a23} is an optionally substituted divalent organic group. R^{a24} in amino group optionally substituted with a hydrocarbon group having 1 or more and 6 or less carbon atoms, or a quaternary ammonium salt group represented by —N⁺R^{a25}R^{a26}R^{a27} Z⁻. R^{a25} , R^{a26} , and R^{a27} are each independently hydrogen atom or a hydrocarbon group having 1 or more and 6 or less carbon atoms. Z⁻ is a counter anion. p is 0 or 1.

In the above formula (a1-ix), when R^{a22} is the group represented by —Y—R^{a23}—R^{a24}, R^{a23} is the optionally substituted divalent organic group. The divalent organic group is not particularly limited, and preferably a divalent hydrocarbon group. A number of carbon atoms in the divalent hydrocarbon group is not particularly limited as long as the objective of the present invention is not impaired. Since the resin (A) is easily prepared and easily available, when R^{a23} is the divalent hydrocarbon group, the number of carbon atoms in the divalent hydrocarbon groups is preferably 1 or more and 20 or less, more preferably 1 or more and 12 or less, particularly preferably 1 or more and 10 or less, and most preferably 1 or more and 6 or less.

The divalent hydrocarbon group as R^{a23} may be an aliphatic group, an aromatic group, or a hydrocarbon group including an aliphatic part and an aromatic part. When the divalent hydrocarbon group is an aliphatic group, the aliphatic group may be a saturated aliphatic group or an unsaturated aliphatic group. In addition, a structure of the aliphatic group may be linear, branched, cyclic, or combination of these structures.

Suitable specific examples of R^{a23} include methylene group, ethane-1,2-diyl group, ethane-1,1-diyl group, propane-1,3-diyl group, propane-1,1-diyl group, propane-2,2-diyl group, n-butane-1,4-diyl group, n-pentane-1,5-diyl group, n-hexane-1,6-diyl group, n-heptane-1,7-diyl group, n-octane-1,8-diyl group, n-nonane-1,9-diyl group, n-decane-1,10-diyl group, o-phenylene group, m-phenylene group, p-phenylene group, naphthalene-2,6-diyl group, naphthalene-2,7-diyl group, naphthalene-1,4-diyl group, and biphenyl-4,4′-diyl group.

When the divalent hydrocarbon group as R^{a23} has a substituent, examples of the substituent include a hydroxy group, an alkoxy group having 1 or more and 6 or less carbon atoms, an aliphatic acyl group having 2 or more and 6 or less carbon atoms, a halogen atom, a nitro group, a cyano group, and the like.

When R^{a24} is the amino group substituted with the alkyl group having 1 or more and 6 or less carbon atoms, Suitable specific examples thereof include amino group, methylamino group, ethylamino group, n-propylamino group, isopropylamino group, n-butylamino group, n-pentylamino group, n-hexylamino group, phenylamino group, dimethylamino group, diethylamino group, di-n-propylamino group, diisopropylamino group, di-n-butylamino group, di-n-pentylamino group, di-n-hexylamino group and diphenylamino group.

When R^{a24} is the quaternary ammonium salt group represented by —N⁺R^{a25}R^{a26}R^{a27}Z⁻, R^{a25}, R^{a26} , and R^{a27} are each independently hydrogen atom or the hydrocarbon group having 1 or more and 6 or less carbon atoms, and Z⁻ is the counter anion. Suitable examples of the hydrocarbon group having 1 or more and 6 or less carbon atoms include methyl group, ethyl group, n-propyl group, isopropyl group, n-butyl group, n-pentyl group, n-hexyl group, and phenyl group. The counter anion as Z⁻ is not particularly limited as long as the anion is a monovalent anion, and a halide ion is preferred. Suitable examples of the halide ion include chloride ion, bromide ion, and iodide ion.

Suitable specific examples of the constituent unit derived from the compound represented by the formula (1-ix) described above include the following constituent units a1-ix-1 to a1-ix-24. From the view point that the monomer compound is easily available, and good surface treatment effect is easily obtained, the constituent units a1-ix-1 to a1-ix-4, a1-ix-17, and a1-ix-18 are preferred.

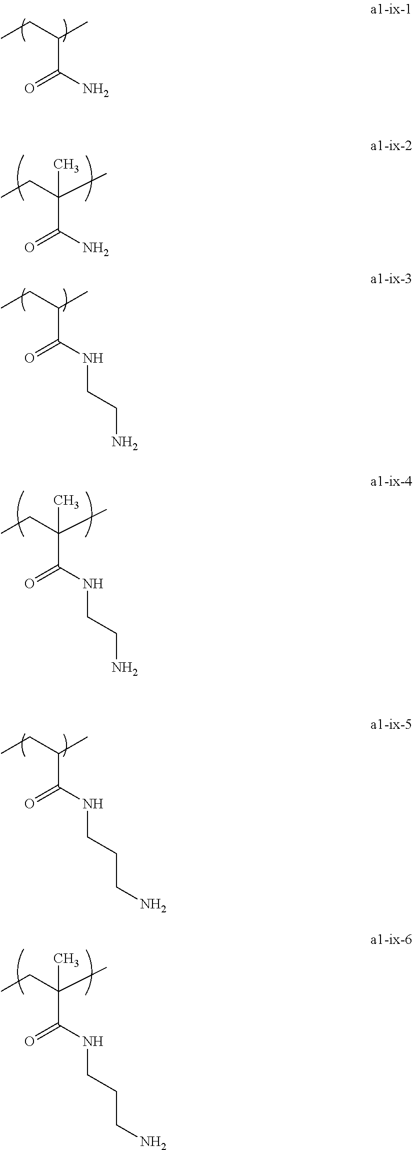

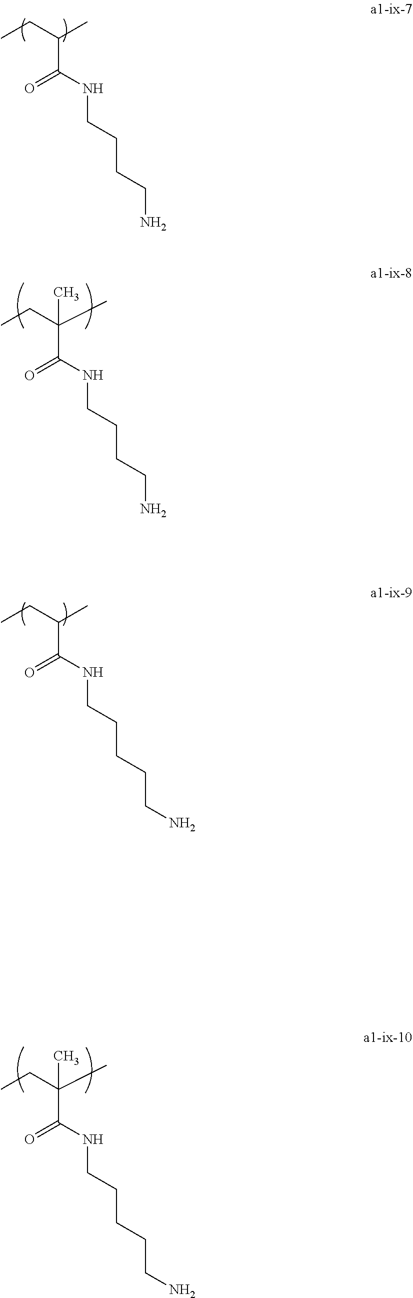

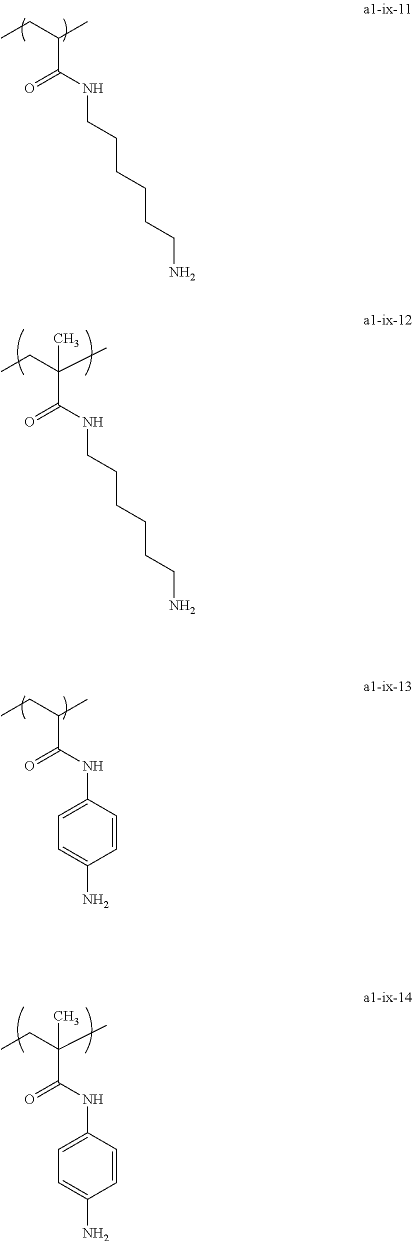

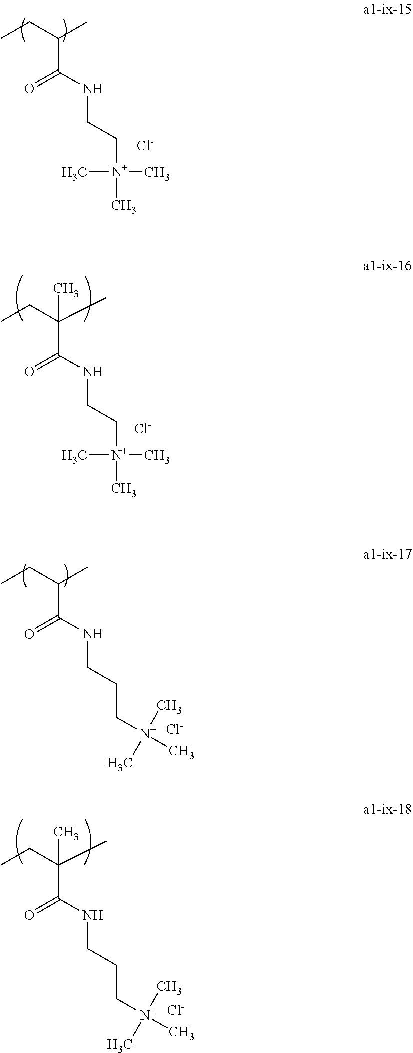

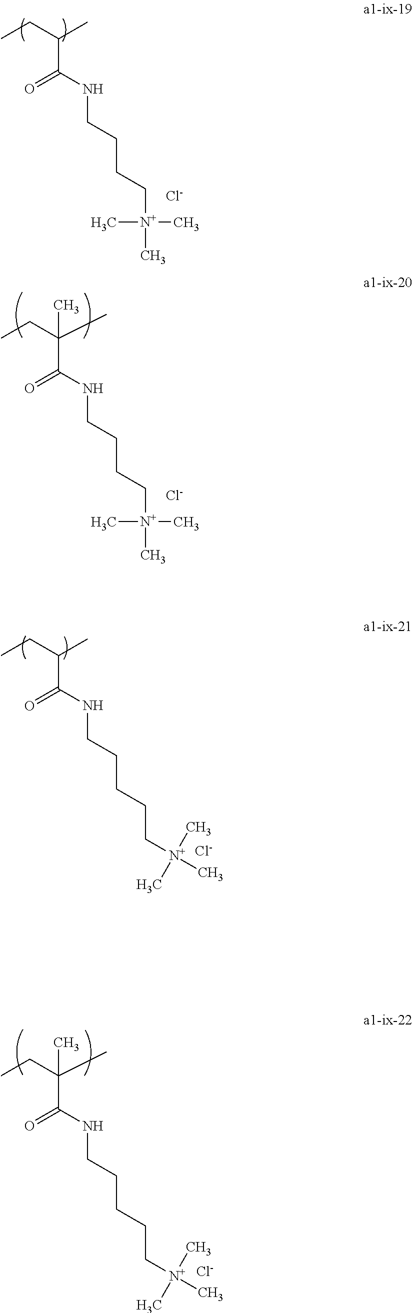

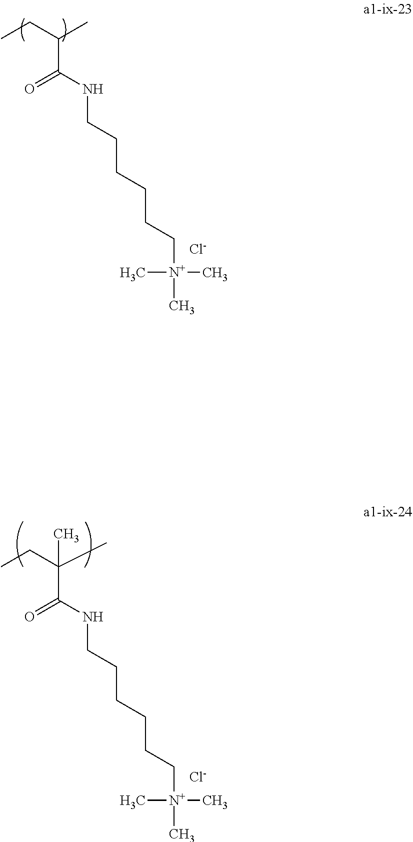

The functional group capable of forming anion is typically a functional group exhibiting Bronsted-acidity, and a salt of a functional group exhibiting Bronsted-acidity. Suitable examples of the functional group exhibiting Bronsted-acidity include carboxylic group, sulfonic acid group, sulfinic acid group, phosphonic acid group, phosphinic acid group, and phenolic hydroxy group.

These Bronsted-acidic groups may form salts with counter cations. The counter cation is not particularly limited, may be an organic cation or an inorganic cation such as metallic ion, and is preferably the metallic ion. As the metallic ion, an alkali metal ion is preferred, and for example, Li⁺, Na⁺, K⁺, and Sr⁺ are preferred.

As the non-betaine monomer having the Bronsted-acidic group as the functional group capable of forming anion, a compound represented by the following formula (a1-x) is preferred.

CH₂═CR^{a31}—(CO))_{b}—R^{a32} (a1-x)

In the formula (a1-x), R^{a31} is hydrogen atom or methyl group. R^{a32} is a hydroxy group or a group represented by —A¹—R^{a33}—R^{a34}. Al is single bond, —O—, or —NH—. R^{a33} is an optionally substituted divalent organic group. R^{a34} is a Bronsted-acidic group. b is 0 or 1. Here, when b is 0, R^{a32} is not hydroxy group, and A¹ is the single bond.

In the above formula (a1-x), R^{a32} is the group represented by —A1—R^{a33}—R^{a34}, R^{a33} is the optionally substituted divalent organic group. The divalent organic group is not particularly limited, and preferably a divalent hydrocarbon group. A number of carbon atoms in the divalent hydrocarbon group is not particularly limited as long as the objective of the present invention is not impaired. Since the resin (A) is easily prepared and easily available, when R^{a33} is the divalent hydrocarbon group, the number of carbon atoms in the divalent hydrocarbon groups is preferably 1 or more and 20 or less, more preferably 1 or more and 12 or less, particularly preferably 1 or more and 10 or less, and most preferably 1 or more and 6 or less.

The divalent hydrocarbon group as R^{a33} may be an aliphatic group, an aromatic group, or a hydrocarbon group including an aliphatic part and an aromatic part. When the divalent hydrocarbon group is an aliphatic group, the aliphatic group may be a saturated aliphatic group or an unsaturated aliphatic group. In addition, a structure of the aliphatic group may be linear, branched, cyclic, or combination of these structures.

Suitable specific examples of R^{a33} include methylene group, ethane-1,2-diyl group, ethane-1,1-diyl group, propane-1,3-diyl group, propane-1,1-diyl group, propane-2,2-diyl group, n-butane-1,4-diyl group, n-pentane-1,5-diyl group, n-hexane-1,6-diyl group, n-heptane-1,7-diyl group, n-octane-1,8-diyl group, n-nonane-1,9-diyl group, n-decane-1,10-diyl group, o-phenylene group, m-phenylene group, p-phenylene group, naphthalene-2,6-diyl group, naphthalene-2,7-diyl group, naphthalene-1,4-diyl group, and biphenyl-4,4′-diyl group.

When the divalent hydrocarbon group as R^{a33} has a substituent, examples of the substituent include a hydroxy group, an alkoxy group having 1 or more and 6 or less carbon atoms, an aliphatic acyl group having 2 or more and 6 or less carbon atoms, a halogen atom, a nitro group, a cyano group, and the like.

As the Bronsted-acidic group as R^{a34}, carboxylic group, sulfonic acid group, sulfinic acid group, phosphonic acid group, phosphinic acid group, and phenolic hydroxy group are preferred, and carboxylic group, sulfonic acid group, and phenolic hydroxy group are more preferred.

When R^{a34} is not the phenolic hydroxy group, the following groups are preferred as the group represented by —R^{a33}—R^{a34}. In the following structure formula, R^{a34} is a Bronsted-acidic group other than phenolic hydroxy group.

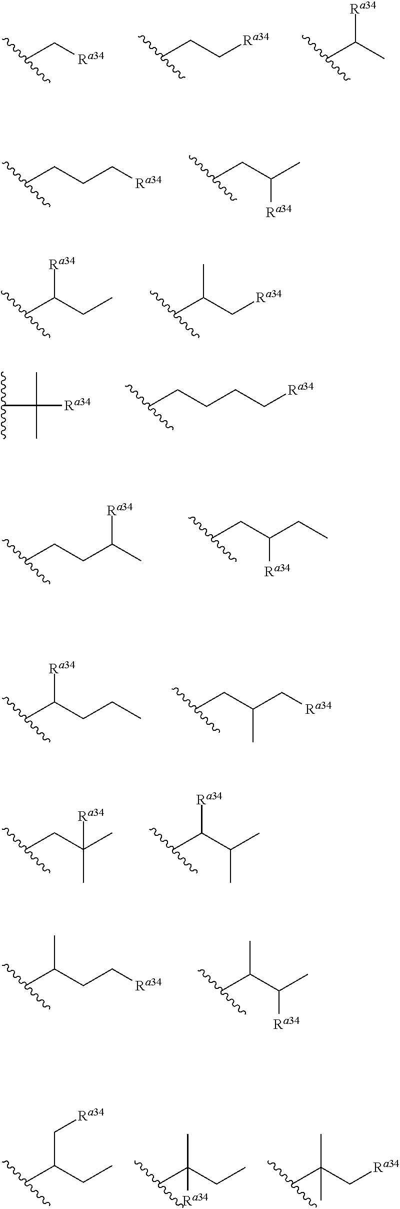

Suitable specific examples of the constituent unit derived from the compound represented by the formula (a1-x) include the following constituent units a1-x-1 to a1-x-20. Among these constituent units, because of easy availability of the monomer compound and the ease of obtaining good surface treatment effect, the constituent units a1-x-1 to a1-x-10, a1-x-19, and a1-x-20 are preferred.

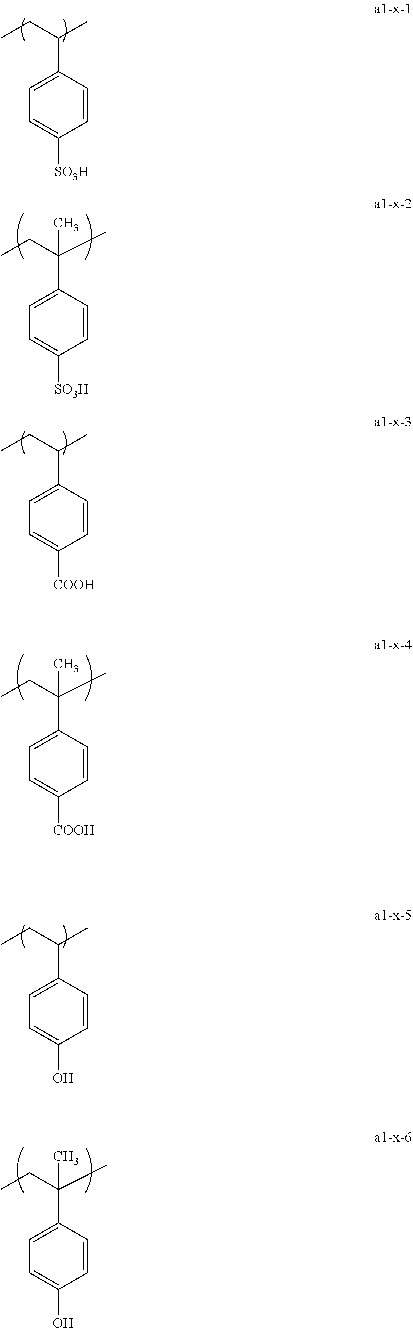

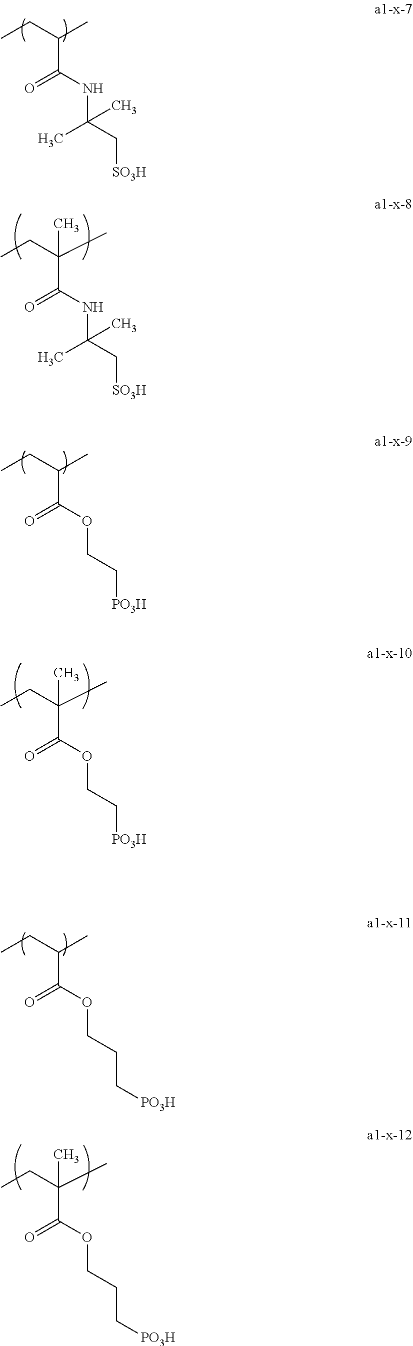

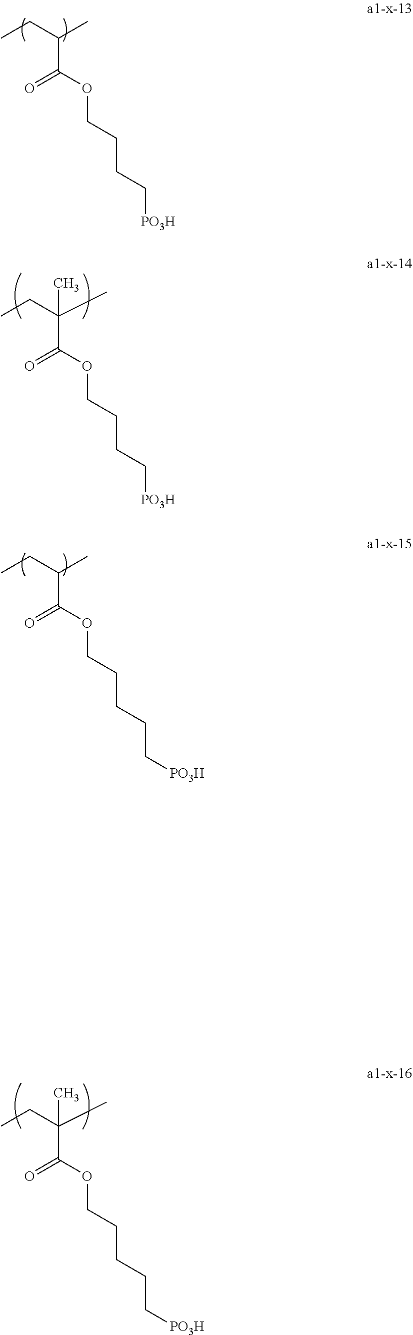

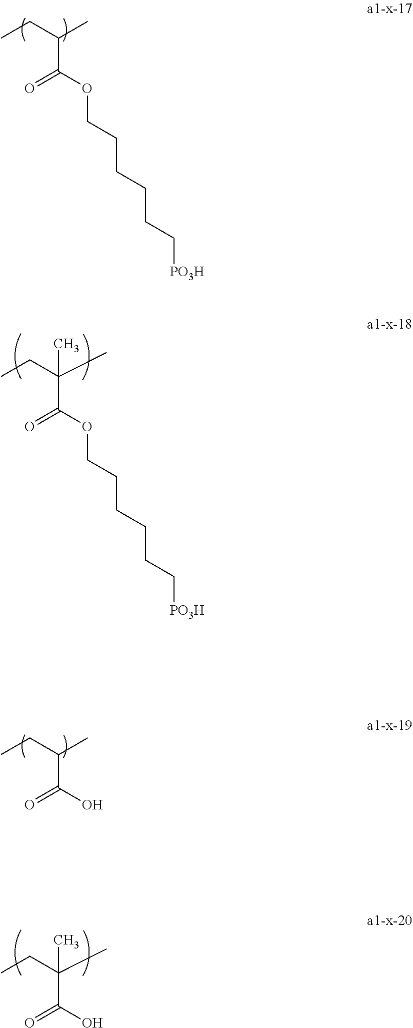

In view of both of adhesiveness of the resin (A) to the surface of the object to be treated, and good surface treatment effect, a ratio of the constituent unit (a1) is preferably 30% by mole or more and 99.9% by mole or less, more preferably 40% by mole or more and 99% by mole or less, and even more preferably 50% by mole or more and 95% by mole or less relative to the all constituent units of the resin (A).

[Constituent Unit (a2)]

The resin (A) includes a constituent unit (a2) derived from a compound represented by the following formula (a-2) for the purpose of adhering well the resin (A) to the surface of the object to be treated.

(R^{A3}—R^{A2})n—X—RA (a-2)

In the formula (a-2), R^{A1} is an organic group having 1 or more ethylenic double bond. R^{A2} is a single bond, or an alkylene group having 1 or more and 10 or less carbon atoms. R^{A3} is a hydrogen atom or a polar group selected from amino group, carboxylic group, mercapto group, hydroxy group, and cyano group. n is 1 or 2. X is a nitrogen-containing heterocyclic group having valency of n+1. When n is 1, R^{A3} is the polar group. When n is 2, at least one of R^{A3}s are the polar group.

The constituent unit (a2) essentially includes the polar group selected form amino group, carboxylic group, mercapto group, hydroxy group, and cyano group. Therefore, when the resin (A) includes the constituent unit (a2) having such polar group, the resin (A) tends to adhere well to the surface of the object to be treated. The aforementioned constituent having carboxylic group as the Bronsted-acidic group naturally corresponds to the constituent unit having carboxylic group.

In the formula (a-2), R^{A2} is the organic group having 1 or more ethylenic unsaturated double bond. Suitable examples of the organic group having 1 or more ethylenic unsaturated double bond include the groups represented by the following formulas (a-2i) to (a-2vii). In the formulas (a-2vi to a-2viii), R^{A01} is an alkenyl group having 1 or more and 10 or less carbon atoms, and R^{A02} is a hydrocarbon group having 1 or more and 10 or less carbon atoms.

—R^{A01} (a-2i)

—NH—R^{A01} (a-2ii)

—N(R^{A01})(R^{A02}) (a-2iii)

—N(R^{A01})₂ (a-2iv)

—O—R^{A01} (a-2v)

—CO—NH—R^{A01} (a-2vi)

—CO—O—R^{A01} (a-2vii)

A number of carbon atoms of the alkenyl group as R^{A01} is preferably 1 or more and 6 or less, and more preferably 1 or more and 4 or less. The alkenyl group as R^{A01} may be a linear alkenyl group or a branched alkenyl group. The hydrocarbon group as R^{A02} may be an aliphatic group, an aromatic group, or a combination of an aliphatic group and an aromatic group. A number of carbon atoms of the hydrocarbon group as R^{A02} is preferably 1 or more and 6 or less, more preferably 1 or more and 4 or less, and further preferably 1 or more and 3 or less.

Suitable specific examples of the organic group having 1 or more ethylenic unsaturated double bond as R^{A1} include alkenyl groups such as vinyl group, 1-propenyl group, 2-n-propenyl group (allyl group), 1-n-butenyl group, 2-n-butenyl group, and 3-n-butenyl group; monoalkenylamino groups such as N-vinylamino group, N-1-propenylamino group, N-allylamino group, N-1-n-butenylamino group, N-2-n-butenyl amino group, and N-3-n-butenylamino group; dialkenylamino groups such as N,N-divinylamino group, N,N-di(1-propenyl)amino group, N,N-diallylamino group, N,N-di(1-n-butenyl)amino group, N,N-di(2-n-butenyl)amino group, and N,N-di(3-n-butenyl)amino group; alkenyloxy groups such as allyloxy group, 2-n-butenyloxy group, and 3-n-butenyloxy group; alkenylaminocarbonyl groups such as vinylaminocarbonyl group, 1-propenylaminocarbonyl group, allylaminocarbonyl group, 1-n-butenylaminocarbonyl group, 2-n-butenylaminocarbonyl group, and 3-n-butenylaminocarbonyl group; alkenyloxycarbonyl groups such as vinyloxycarbonyl group, 1-propenyloxycarbonyl group, allyloxycarbonyl group, 1-n-butenyloxycarbonyl group, 2-n-butenyloxycarbonyl group, and 3-n-butenyloxycarbonyl group; (meth)acryloyl group-containing groups such as acryloyl group, methacryloyl group, acryloyloxy group, methacryloyloxy group, acryloylamino group, and methacryloylamino group. Among these groups, vinyl group, allyl group, N,N-diallylamino group, allyloxy group, acryloyl group, methacryloyl group, acryloyloxy group, and methacryloyloxy group are preferred, and N,N-diallylamino group is more preferred.

In the formula (a-2), R^{A2} is a single bond or an alkylene group having 1 or more and 10 or less carbon atoms. A number of carbon atoms in the alkylene group is preferably 1 or more and 6 or less, more preferably 1 or more and 4 or less, and further preferably 1 or more and 3 or less. Specific examples of the alkylene group having 1 or more and 10 or less carbon atoms include methylene group, ethane-1,2-diyl group, ethane-1,1-diyl group, propane-1,3-diyl group, propane-1,1-diyl group, propane-2,2-diyl group, n-butane-1,4-diyl group, n-pentane-1,5-diyl group, n-hexane-1,6-diyl group, n-heptane-1,7-diyl group, n-octane-1,8-diyl group, n-nonane-1,9-diyl group, and n-decane-1,10-diyl group. Among these alkylene groups, methylene group, ethane-1,2-diyl group, and propane-1,3-diyl group are preferred, and methylene group, and ethane-1,2-diyl group are more preferred.

In the formula (a-2), X is a nitrogen-containing heterocyclic group of valency of n+1. n is 1 or 2. The nitrogen-containing heterocyclic group may be an aromatic group or an aliphatic group. The nitrogen-containing heterocycle may be a monocycle or a condensed polycycle in which a monocyclic nitrogen-containing heterocycle bonds to one or more monocycles selected from a monocyclic aromatic hydrocarbon ring and a monocyclic nitrogen-containing heterocycle. In addition, the nitrogen-containing heterocycle may be a ring in which two or more rings selected from a monocyclic nitrogen-containing heterocycle and a condensed polycyclic nitrogen-containing heterocycle bond together through a single bond.

In the formula (a-2), a group represented by R^{A1}, and a group represented by R^{A3}—R^{A2}— may bond to carbon atom as a ring-constituting atom or nitrogen atom as a ring-constituting atom on the nitrogen-containing heterocyclic group represented by X.

Examples of the nitrogen-containing heterocycle giving X include 5-membered ring such as a pyrrolidine ring, a pyrazolidine ring, an imidazolidine ring, a triazolidine ring, a tetrazolidine ring, a pyrroline ring, a pyrazoline ring, an imidazoline ring, a triazoline ring, a tetrazoline ring, a pyrrole ring, a pyrazole ring, an imidazole ring, a triazole, and a tetrazole ring; a nitrogen-containing 6-membered ring such as a piperidine ring, a piperideine ring, a piperazine ring, a triazinane ring, a tetradinane ring, a pentazinane ring, a pyridine ring, a pyridazine ring, a pyrimidine ring, a pyrazine ring, a triazine ring, a tetrazine ring, and a pentazine ring; a nitrogen-containing 7-membered ring such as an azepane ring, a diazepane ring, a triazepane ring, a tetrazepam ring, an azepine ring, a diazepine ring, and a triazepine ring; a nitrogen-containing condensed polycycle such as an indole ring, an indolenine ring, an indoline ring, an isoindole ring, an isoindolenine ring, isoindoline ring, a benzimidazole ring, an indolizine ring, a purine ring, an indolizidine ring, a benzodiazepine ring, a quinoline ring, an isoquinoline ring, a quinolizidine ring, a quinoxaline ring, a cinnoline ring, a quinazoline ring, a phthalazine ring, a naphthyridine ring, and a pteridine ring; polycycles in which 2 or more rings selected from these nitrogen-containing heterocycles are bonded via single bond(s). As X derived from these nitrogen-containing heterocycle, from the viewpoint that the resin (A) has good adhesiveness to the surface of the object, a divalent or trivalent group including a nitrogen-containing 6-membered ring is preferred, a divalent or trivalent group including a triazine ring is more preferred, and 1,3,5-triazine-2,4-diyl group and 1,3,5-triazine-2,4,6-triyl group are even more preferred.

Suitable specific examples of the divalent or trivalent nitrogen-containing heterocyclic group include the following groups.

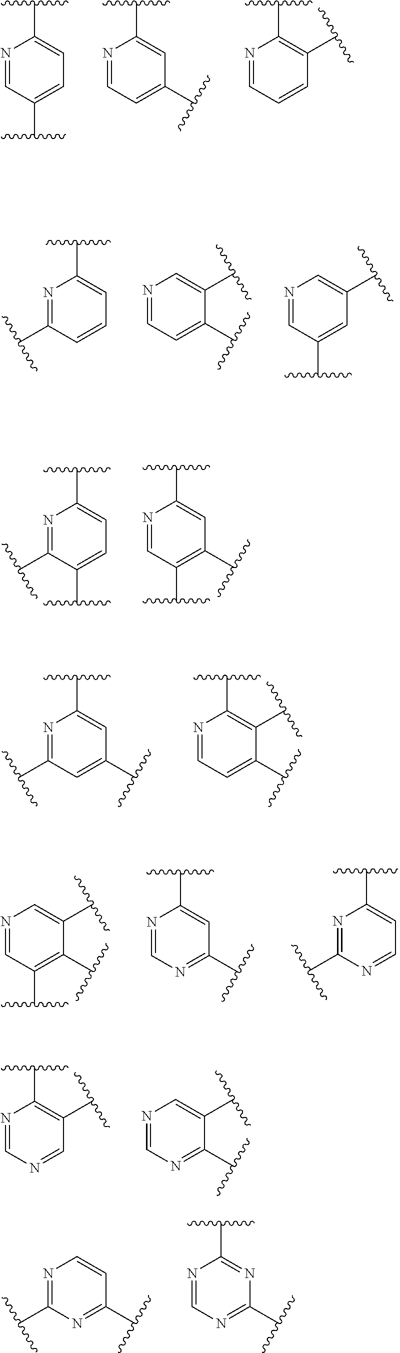

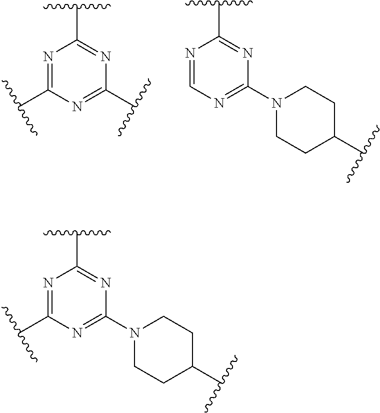 [SMILES: *c1nc(*)nc(*)n1 ; *c1ncnc(N2CCC(*)CC2)n1 ; *c1nc(*)nc(N2CCC(*)CC2)n1]

Suitable specific examples of the compound represented by the formula (a-2) includes the following compounds.

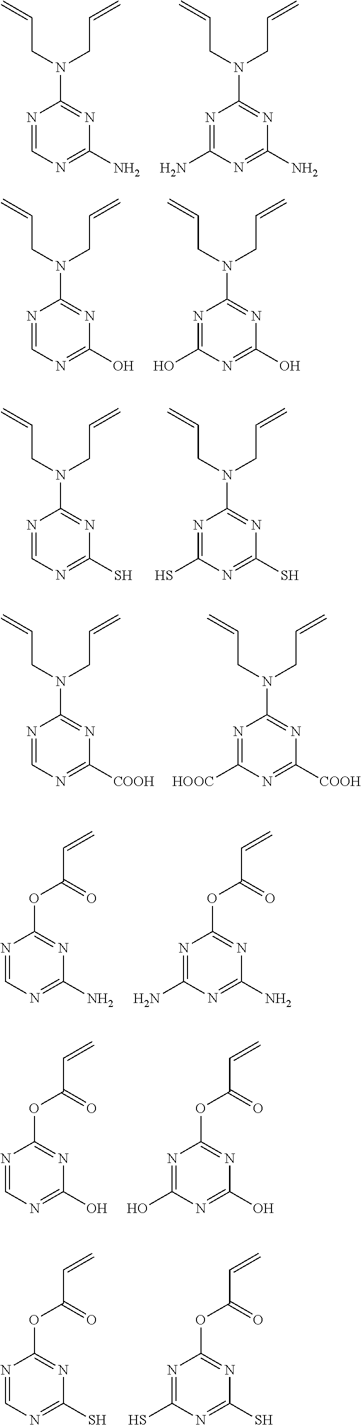

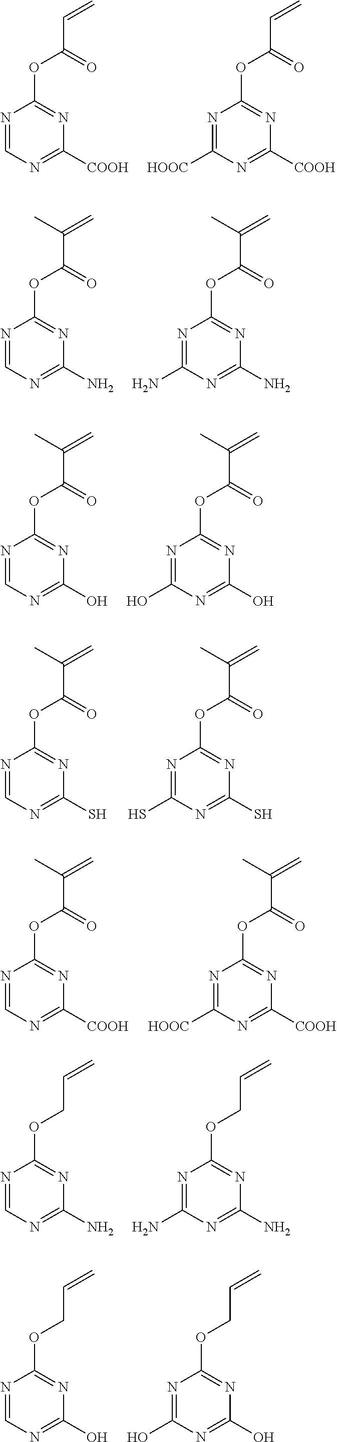

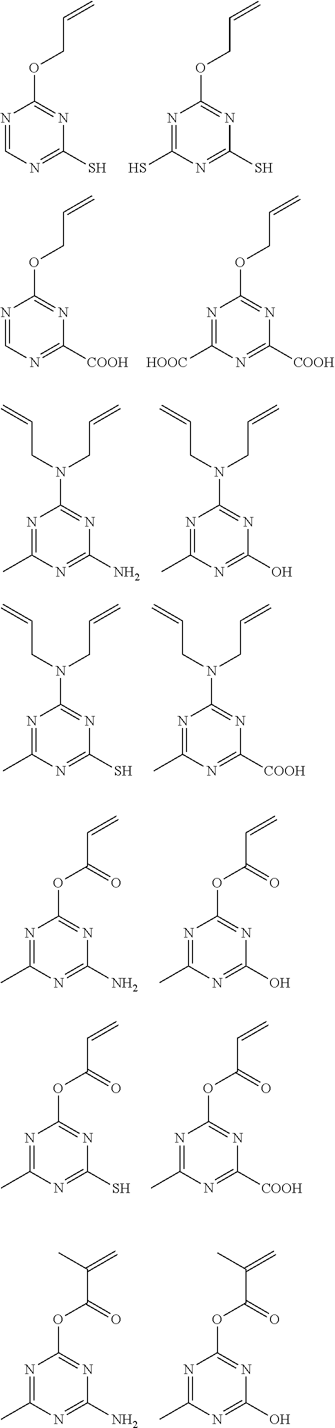

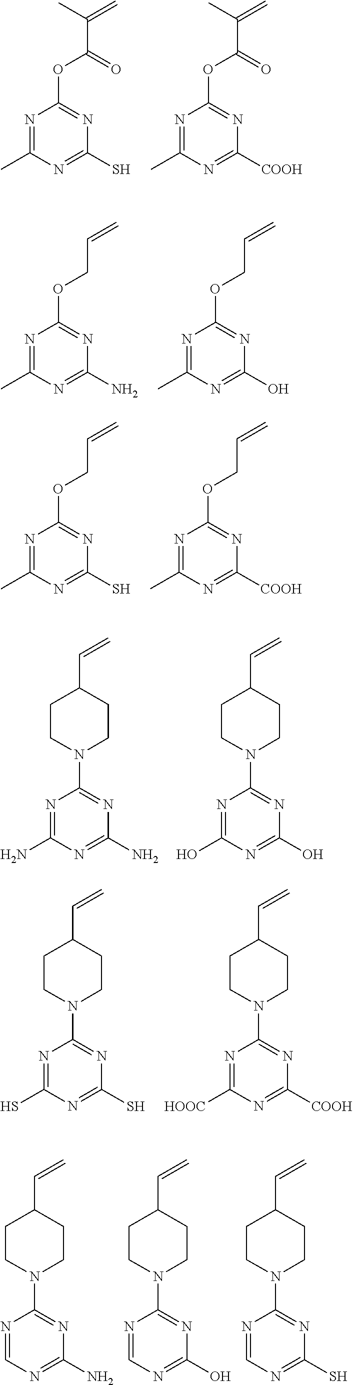

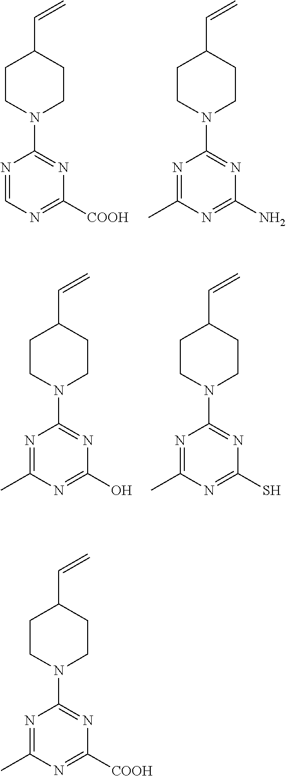

Among the above compounds, the following compounds are preferred.

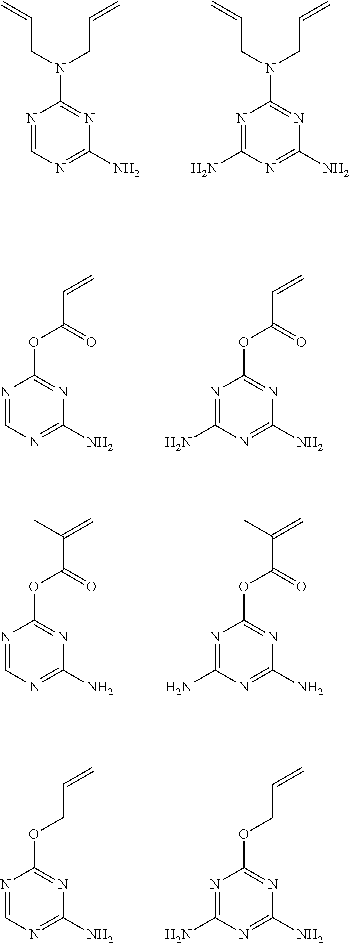

In view of both of adhesiveness of the resin (A) to the surface of the object to be treated, and good surface treatment effect, a ratio of the constituent unit (a2) is preferably 0.1% by mole or more and 70% by mole or less, more preferably 1% by mole or more and 60% by mole or less, and even more preferably 5% by mole or more and 50% by mole or less relative to all constituent units of the resin (A).

[Constituent Unit (a3)]

The resin (A) may include a constituent unit (a3) other than the constituent unit (a1) and the constituent unit (a2) as long as the objective of the present invention is not impaired. Examples of compounds giving the constituent unit (a3) in the resin (A) include, for example, methyl (meth)acrylate, ethyl (meth)acrylate, isopropyl (meth)acrylate, n-propyl (meth)acrylate, n-butyl (meth)acrylate, isobutyl (meth)acrylate, a tert-butyl (meth)acrylate, n-pentyl (meth)acrylate, isopentyl (meth)acrylate, phenyl (meth)acrylate, N-methyl (meth)acrylamide, N-ethyl (meth)acrylamide, N-n-propyl (meth)acrylamide, N-isopropyl (meth)acrylamide, N-n-butyl (meth)acrylamide, N-n-pentyl (meth)acrylamide, N-isopentyl (meth)acrylamide, N-phenyl (meth)acrylamide, N,N-dimethyl (meth)acrylamide, N,N-diethyl (meth)acrylamide, N,N-di-n-propyl (meth)acrylamide, N,N-di-n-butyl (meth) acrylamide, N,N-di-n-pentyl (meth) acrylamide, styrene, α-methylstyrene, β-methylstyrene, o-methylstyrene, m-methylstyrene, p-methylstyrene, chlorostyrene, methyldiallylamine, ethyldiallyamine, triallylamine, and the like.

### (Synthesis Method of Resin (A))

The resin (A) can be prepared by polymerizing the compound giving the constituent unit (a1) and the compound represented by the formula (a-2) giving the constituent unit (a2) according to well-known method. As a preferred method, a method of radically polymerizing monomers giving constituents constituting the resin (A) in the presence of a polymerization initiator. As the polymerization initiator, for example, azo polymerization initiator is exemplified. As such polymerization initiator, 2,2′-azobis(2-methylproipionamidine) dihydrochloride, 2,2′-azobis[2-(phenylamidino)propane] dihydrochloride, 2,2′-azobis{2-[N-(4-chlorophenyl)amidino]propane} dihydrochloride, 2,2′-azobis{2-[N-(4-hydroxyphenyl)amidino]propane} dihydrochloride, 2,2′-azobis[2-(N-benzylamidino)propane] dihydrochloride, 2,2′-azobis[2-(N-allylamidino)propane] dihydrochloride, 2,2′-azobis(2-amidinopropane) dihydrochloride, 2,2′-azobis{2-[N-(4-hydroxyethyl)amidino]propane} dihydrochloride, 2,2-azobis[2-(5-methyl-2-imidazoline-2-yl)propane] dihydrochloride, 2,2-azobis[2-(2-imidazoline-2-yl)propane] dihydrochloride, 2,2-azobis[2-(4,5,6,7-tetrahydro-1H-1,3-diazepine-2-yl)propane] dihydrochloride, 2,2-azobis[2-(3,4,5,6-tetrahydropyrimidine-2-yl)propane] dihydrochloride, 2,2-azobis[2-(5-hydroxy-3,4,5,6-tetrahydropyrimidine-2-yl)propane] dihydrochloride, 2,2-azobis{2-[1-(2-hydroxyethyl)-2-imidazoline-2-yl]propane} dihydrochloride, and 2,2-azobis[2-(2-imidazoline-2-yl)propane] are exemplified. These polymerization initiators may be used singly or two or more thereof may be used in combination. Amount or the initiator is not particularly limited as long as polymerization reaction proceeds well. Amount of the initiator is preferably 0.1% by mole or more and 20% by mole or less, more preferably 0.1% by mole or 15% by mole or less relative to a number of moles of all monomers.

A ratio of the mass of the resin (A) is not particularly limited, preferably 0.1% by mass or more and 5% by mass or less, more preferably 0.1% by mass or more and 3.0% by mass or less, and even more preferably 0.1% by mass or more and 1.5% by mass or less relative to the mass of the surface treatment liquid.

### <Electrolyte (B)>

The surface treatment liquid may include an electrolyte (B). When the surface treatment liquid includes the electrolyte (B), the resin (A) is easily dissolved in the surface treatment liquid. It should be noted that the electrolyte (B) is a material other than the resin (A). For example, a polymer compound which corresponds to the resin (A) and can be ionized in the surface treatment liquid is defined as not the electrolyte (B) but the resin (A).

A type of the electrolyte (B) is not particularly limited as long as it is a substance that does not decompose the resin (A). A type of the electrolyte (B) is not particularly limited. The electrolyte (B) may be a substance such as hydrochloric acid, sodium chloride, and potassium chloride generally recognized as a strong electrolyte or a substance such as anionic surfactant (for example, sodium dodecyl sulfonate, and the like), anionic surfactant (for example, benzalkonium chloride, and the like) generally recognized as a weak electrolyte.

In view of ease of availability, being inexpensive, and the like, suitable examples of the electrolyte (B) include sodium chloride, potassium chloride, sodium perchlorate, potassium perchlorate, sodium hydroxide, potassium hydroxide, perchloric acid, hydrochloric acid, and sulfuric acid.

A content of the electrolyte (B) is not particularly limited as long as the objective of the present invention is not impaired, and appropriately determined taking into consideration solubility in the surface treatment liquid, and the like. For example, the content of the electrolyte (B) is preferably 0 parts by mass or more and 700 parts by mass or less, and more preferably 0 parts by mass or more and 600 parts by mass or less, and even more preferably 0 parts by mass or more and 500 parts by mass or less relative to 100 parts by mass of the resin (A).

### [Solvent (S)]

The surface treatment liquid includes a solvent (S). The solvent (S) may be water, an organic solvent, or an aqueous solution of the organic solvent. From the viewpoint of the solubility of the resin (A), the safety of working for the surface treatment, low costs and the like, the solvent (S) is preferably water. Suitable examples of the organic solvent used as the solvent (S) include an alcohol. As the alcohol, an aliphatic alcohol is preferred, and the alcohol having 1 or more and 3 or less carbon atoms is more preferred. Specifically, methanol, ethanol, n-propyl alcohol, and isopropyl alcohol (IPA) are exemplified, and methanol, ethanol, and isopropyl alcohol are preferred. The alcohols may be used singly or two or more thereof may be used in combination.

The content of water in the solvent (S) is preferably 50% by mass or more, more preferably 80% by mass or more and particularly preferably 100% by mass.

### [Other Component]

The surface treatment liquid may include various additives as long as the objective of the present invention is not impaired. Examples of such additives include a thermal polymerization inhibitor, photo polymerization inhibitor, an antioxidant, an ultraviolet absorber, a colorant, a defoamer, a viscosity modifier and the like. A content of these additives is appropriately determined in consideration of an amount in which these additives are ordinarily used.

### «Surface Treatment Method»

A surface treatment method is not particularly limited as long as the resin (A) can bond or adhere to the surface of the object to be treated by the method so that an affinity of the surface of the object to be treated for water can be adjusted to the desired degree. Typically, the surface treatment method includes forming a coating film on the surface of the object to be treated by applying the aforementioned surface treatment liquid. Here, there is no need to form the coating film uniformly on the entire surface of the object to be treated, as long as an affinity of the surface of the object to be treated for water can be adjusted to the desired degree. The surface treatment method preferably further includes rinsing the surface of the object to be treated by a rinse liquid after application of the surface treatment liquid.

Herein, forming the coating film on the surface of the object to be treated by applying the surface treatment liquid is also described as “application step”. In addition, rinsing the surface of the object to be treated by a rinse liquid after application of the surface treatment liquid is also described as “rinsing step”. Hereinafter, application step, rinsing step, and surface treatment liquid will be described in detail.

### <Application Step>

In the application step, the coating film is formed by applying the aforementioned surface treatment liquid on the surface of the object to be treated. The application method is not particularly limited. Specific examples of the application method include a spin coating method, a spraying method, a roller coating method, an immersion method and the like. In a case where the object to be treated is a substrate, the spin coating method is preferable as the application method since it is easy to evenly form a coating film having a uniform film thickness on the surface of the substrate.

The material of the surface of the object to be treated to which the surface treatment liquid is applied is not particularly limited and may be an organic material or an inorganic material. Examples of the organic material include a variety of resin materials such as a polyester resin such as a PET resin or a PBT resin, a variety of nylons, a polyimide resin, a polyamide-imide resin, a polyolefin such as polyethylene or polypropylene, polystyrene, a (meth)acrylic resin, a cycloolefin polymer (COP), a cycloolefin copolymer (COC) and a silicone resin (for example, polyorganosiloxane such as polydimethylsiloxane (PDMS)). In addition, a photosensitive resin component that is included in a variety of resist materials as well as an alkali-soluble resin component are also preferable as the organic material. Examples of the inorganic material include glass, silicon and a variety of metals such as copper, aluminum, iron and tungsten. The metals may be alloys.

The shape of the object to be treated is not particularly limited. The object to be treated may be flat or a three-dimensional shape, for example, a spherical shape or a columnar shape.

There is a case that the object to be treated is exposed to chemicals such as a detergent. When the coating film adhering to the surface of the object to be treated is exposed to various chemicals, depending on types of chemicals, there is a concern that the surface treatment effect due to the coating film is significantly impaired. However, by using the aforementioned surface treatment liquid, decreasing of the surface treatment effect when the surface of the object to be treated is come into contact with various chemical solution can be suppressed. Therefore, when articles consisting of glasses or transparent resins included in the object to be treated, which is often exposed to the chemicals such as the detergent, such as windows, mirrors, furniture, optical devices (for example, devices equipped with lens) are used as the object to be treated, resistance to chemicals as the surface treatment effect is significantly achieved.

After the surface treatment liquid is applied to the surface of the object to be treated, at least part of the solvent (S) may be removed from the coating film consisting of the surface treatment liquid as necessary by a well-known drying method.

The film thickness of the coating film that is formed in the application step is not particularly limited. The thickness of the coating film that is formed in the application step is, for example, preferably 1 μm or less, more preferably 300 nm or less and even more preferably 100 nm or less.

In the application step, the thickness of the coating film can be adjusted by adjusting a solid content of the surface treatment liquid, conditions for application, and the like.

### <Rinsing Step>

In the rinsing step, the surface of the object to be treated is rinsed with a rinse liquid after applying the surface treatment liquid. By rinsing, the coating film formed on the object to be treated can be thinner. The rinse liquid is not particularly limited as long as the coating film with desired thickness can be formed. As the rinse liquid, water, an organic solvent and an aqueous solution of an organic solvent can be used. As the rinse liquid, water is preferred. Method for rinsing the coating film is not particularly limited. Typically, rinse is carried out by contacting the rinse liquid to the coating film in the same manner as the aforementioned method of application.

It should be noted that a part or all of the solvent (S) included in coating film may be removed by heating the coating film. A heating temperature is not particularly limited as long as it does not cause degradation or decomposition of the object to be treated or the resin (A). As a typical heating temperature, a temperature in a range of around 50° C. or higher and 300° C. or lower is exemplified. Heating time is not particularly limited, for example, preferably 5 seconds or longer and 24 hours or shorter, and preferably 10 seconds or longer and 6 hours or shorter.

The film thickness of the coating film after the rinsing is, for example, preferably 10 nm or less, more preferably 0.1 nm or more and 10 nm or less, still more preferably 0.1 nm or more and 8 nm or less, far still more preferably 0.5 nm or more and 5 nm or less and particularly preferably 0.5 nm or more and 3 nm or less.

The thickness of the coating film can be adjusted by adjusting a solid content of the surface treatment liquid, conditions for application, an amount of the rinse liquid, a type of the rinse liquid, a temperature of the rinse liquid, and the like.

After the rinsing, the object to be treated is dried as necessary and then preferably used in a variety of uses.

### EXAMPLES

Hereinafter, the present invention will be more specifically described by showing examples, but the scope of the present invention is not limited to these examples.

### Examples 1 to 8 and Comparative Examples 1 to 4

In Examples and Comparative Examples, following A1-1 to A1-5 were used as the monomer compound giving the aforementioned constituent unit (a1). The following A2-1 was used as a monomer giving the constituent unit (a2). The following A3-1 was used as a monomer compound giving the constituent unit (a3).

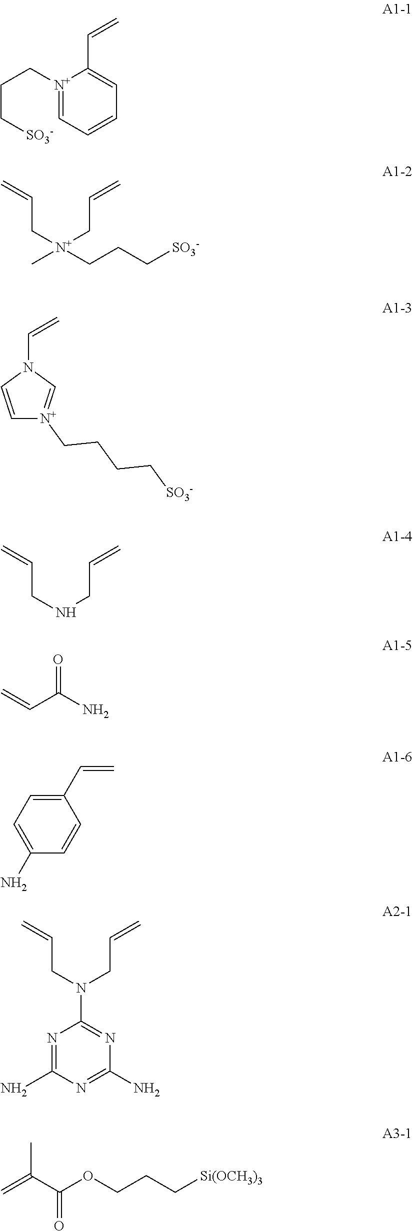

In Examples 1 to 8 and Comparative Examples 1 to 4, monomers of a composition shown in Table 1 are solely polymerized or copolymerized to obtain the resin. Specifically, monomer compounds and polymerization initiators of the types and amounts (mmol) shown in Table 1 were made into an aqueous solution with a monomer concentration of 40% by mass, and radical polymerization was carried out at 80° C. for 4 hours under a nitrogen atmosphere to obtain an aqueous solution or suspension of the resin.

2,2′-azobis(2-methylpropionamidine) dihydrochloride was used as the polymerization initiator.

Sodium chloride in a mass of four times the mass of the resin was added to the aqueous solutions or suspensions of the resins obtained in each of Examples and Comparative Examples to obtain the surface treatment liquids. The surface treatment liquid was clarified by adding sodium chloride. Following evaluations are carried out using the obtained surface treatment liquids.

### <Contact Angle of Water>

The surface treatment liquid was applied onto a silicon wafer by spin coating under conditions of 1000 rpm and 60 seconds, and the wafer was heated at 80° C. for 60 seconds. Subsequently, a coating film consisting of the above resin with a thickness of monoatomic layer level was formed. A pure water drop (2.0 μL) was added dropwise to the surface-treated surface of the silicon wafer using DropMaster 700 (manufactured by Kyowa Interface Science Co., Ltd.), and the contact angle of the water was measured as a contact angle after 10 seconds from the dropwise addition. Average value of the contact angle at three points on the silicon wafer was shown in Table 1. It should be noted the contact angle of the water on the untreated silicon wafer is 13.8°.

### <Chemical Solution Resistance>

The silicon wafer surface-treated by the same method as for the measurement of the contact angle of water was immersed in an aqueous solution of sodium dodecyl sulfate at a concentration of 1% by mass, which is an anionic chemical solution, for 1 minute. Thereafter, the surface of the silicon wafer was washed with pure water by showering for 1 minute. The contact angle of water on the surface-treated surface of the washed silicon wafer was measured according to the above-described method. Contac angles of the water measured are shown in Table 1. The tests for chemical solution resistance to cationic chemicals solution for the silicon wafer surface-treated with the surface treatment liquid of Example 4, the silicon wafer surface-treated with the surface treatment liquid of Example 6, the silicon wafer surface-treated with the surface treatment liquid of Example 7, and the silicon wafer surface-treated with the surface treatment liquid of Comparative Example 2, were carried out in the same manner as the above method except for changing the aqueous solution of sodium dodecyl sulfonate at concentration of 1% by mass to an aqueous solution of dodecyltrimethylammonium chloride at concentration of 1% by mass which is a cationic chemical solution. Contact angles of the silicon wafers after immersion in the cationic chemical solution and washing with pure water are shown in the table 1. It should be noted that the above test regarding the cationic chemical solution was also carried out with respect to the silicon wafer not surface-treated. As a result, the contact angle of water of the silicon wafer after immersion in the cationic chemical solution and washing with pure water is 71.2°. There are active Si—OH groups on the untreated silicon wafer. Therefore, it is thought that dodecyltrimethylammonium chloride is adsorbed or bonded to the surface of the silicon wafer, resulting in a larger value of contact angle of water.

### <N1s Intensity>

The surface of the silicon wafer surface-treated by the same method as for the measurement of the contact angle of water was measured by X-ray photoelectron spectroscopy (XPS) to determine the intensity of the N1s peak, which is attributed to the nitrogen atoms included in the resin. The higher the value of the intensity of N1s peak, the better the resin is bonded to the substrate.

TABLE 1

| | | Examples | | | | | | | | Comparative Examples | | | |
|---|---|---|---|---|---|---|---|---|---|---|---|---|---|
| | | 1 | 2 | 3 | 4 | 5 | 6 | 7 | 8 | 1 | 2 | 3 | 4 |
| Monomer | A1-1 | 45.9 | 25.5 | — | — | — | — | — | — | 48.5 | — | 36.4 | 45.9 |
| compounds | A1-2 | — | — | 45.9 | 25.5 | — | — | — | — | — | — | — | — |
| (mmol) | A1-3 | — | — | — | — | 45.9 | 25.5 | — | — | — | — | — | — |
| | A1-4 | — | — | — | — | — | — | 41.7 | — | — | 40.4 | — | — |
| | A1-5 | — | — | — | — | — | — | — | 56.3 | — | — | — | — |
| | A1-6 | — | — | — | — | — | — | — | — | — | — | 1.9 | — |
| | A2-1 | 5.1 | 25.5 | 5.1 | 25.5 | 5.1 | 25.5 | 2.2 | 1.4 | — | — | — | — |
| | A3-1 | — | — | — | — | — | — | — | — | 2.5 | 2.1 | — | — |

| | | | | | | | | | | | | |
|---|---|---|---|---|---|---|---|---|---|---|---|---|
| Polymerization | 0.51 | 0.51 | 0.51 | 0.51 | 0.51 | 0.51 | 0.51 | 0.51 | 0.51 | 0.51 | 0.51 | 0.51 |
| initiator | | | | | | | | | | | | |
| (mmol) | | | | | | | | | | | | |
| Contact angle | 4.6 | 13.2 | 4.4 | 15.6 | 4.4 | 14.2 | 10.6 | 12.5 | 8.7 | 15.7 | 20.1 | 41.2 |
| of water (°) | | | | | | | | | | | | |
| Chemical | 4.5 | 13.2 | 4.6 | 16.0 | 4.3 | 14.2 | 68.2 | 13.1 | 9.1 | 71.3 | 23.5 | 45.4 |
| solution | | | | | | | | | | | | |
| resistance (°) | | | | | | | | | | | | |
| (Anionic | | | | | | | | | | | | |
| solution) | | | | | | | | | | | | |
| Chemical | — | — | — | 15.3 | — | 13.8 | 10.3 | — | — | 16.0 | — | — |
| solution | | | | | | | | | | | | |
| resistance (°) | | | | | | | | | | | | |
| (Cationic | | | | | | | | | | | | |
| solution) | | | | | | | | | | | | |
| N1s Intensity | 21041 | 21441 | 21451 | 21013 | 23114 | 24315 | 21477 | 21398 | 8215 | 4321 | 6641 | 1201 |
| (Counts) | | | | | | | | | | | | |

According to Examples 1 to 8, from the measurement results of N1s intensity, it is found that the resin in the surface treatment liquid bonds well to the surface of the object to be treated in case that the surface treatment is carried out by using the surface treatment liquid including the resin (A) having the constituent unit (a1) and the constituent unit (2) described above. In addition, from comparison between Examples 1 to 6 and Example 7, it is found that the contact angle of water is not change, and the effect of the surface treatment hardly decrease, when the surface-treated surface of the object to be treated comes in to contact with anionic or cationic impurities, in case that the resin (A) has the constituent unit derived from the betaine monomer as the constituent unit (a1). According to Comparative Example 1 and Comparative Example 2, from the measurement results of the N1s intensity, it is found that the adhesiveness of the resin (A) to the surface of the object to be treated is significantly poorer than that of the resin (A) including the aforementioned constituent unit (a1) and constituent unit (a2), even if the resin (A) includes a constituent unit having the hydrolysable silyl group which is generally recognized as a group having a bonding property to the surface of the object to be treated.

1. A surface treatment liquid comprising a resin (A), and a solvent (S), wherein the resin (A) comprises a constituent unit (a1) derived from a compound having a hydrophilic group and an ethylenic double bond and a constituent unit (a2) derived from a compound represented by formula (a-2):
(R^{A3}—R^{A2})ₙ—X—R^{A1} (a-2)
wherein, in the formula (a-2), R^{A1} is an organic group having one or more ethylenic double bond, R^{A2} is a single bond, or an alkylene group having 1 or more and 10 or less carbon atoms, R^{A3} is a hydrogen atom or a polar group selected from amino group, carboxylic group, mercapto group, hydroxy group, and cyano group, n is 1 or 2, X is a nitrogen-containing heterocyclic group having valency of n+1, when n is 1, R^{A3} is the polar group, and when n is 2, at least one of R^{A3}s are the polar group.

2. The surface treatment liquid according to claim 1, wherein the constituent unit (a1) is derived from a betaine monomer having a cationic group, an anionic group, and the ethylenic double bond.

3. The surface treatment liquid according to claim 2, wherein the cationic group is a quaternary nitrogen cation group.

4. The surface treatment liquid according to claim 2, wherein the anionic group is sulfonic acid anion group, phosphonic acid anion group, or carboxylic acid anion group.

5. The surface treatment liquid according to claim 1, wherein the R^{A1} is N,N-diallyamino group.

6. The surface treatment liquid according to claim 1, wherein the X is 1,3,5-triazine-2,4-diyl group, or 1,3,5-triazine-2,4,6-triyl group.

7. The surface treatment liquid according to claim 1 , wherein a ratio of the constituent unit (a2) is 0.1% by mol or more and 70% by mol or less relative to all constituent units in the resin (A).

8. The surface treatment liquid according to claim 1, wherein a content of the resin (A) in the surface treatment liquid is 0.1% by mass or more and 5% by mass or less.

9. The surface treatment liquid according to claim 1 , further comprising an electrolyte (B).

10. The surface treatment liquid according to claim 1, wherein the solvent (S) comprises water.

11. A surface treatment method to a surface of an object comprising:
forming a coating film on the surface of the object by applying the surface treatment liquid according to claim 1.
